(19) United States
(12) Patent Application Publication
(10) Pub. No.: US 2023/0364244 A1
(43) Pub. Date: Nov. 16, 2023

(54) DENDRIMER COMPOSITIONS AND METHODS FOR DRUG DELIVERY TO INJURED KIDNEY

(71) Applicant: Ashvattha Therapeutics, Inc., Redwood City, CA (US)

(72) Inventors: Jeffrey L. Cleland, Redwood City, CA (US); Kannan Rangaramanujam, Baltimore, MD (US); Sujatha Kannan, Redwood City, CA (US); Rishi Sharma, Redwood City, CA (US); Santiago Appiani La Rosa, Redwood City, CA (US)

(73) Assignee: Ashvattha Therapeutics, Inc., Redwood City, CA (US)

(21) Appl. No.: 18/016,262

(22) PCT Filed: Jul. 16, 2021
(86) PCT No.: PCT/US2021/042097

§ 371 Date: Jan. 13, 2023

Related U.S. Application Data
(60) Provisional application No. 63/053,228, filed on Jul. 17, 2020.

(51) Int. Cl. A61K 47/59; A61K 47/60; A61K 47/65; A61K 45/06; A61P 3/10; A61P 13/12

(52) U.S. Cl. CPC A61K 47/595; A61K 47/60; A61K 47/65; A61K 45/06; A61P 3/10; A61P 13/12

(57) ABSTRACT

In some aspects, the disclosure provides methods of treating or preventing one or more symptoms of a kidney injury, disease or disorder in a subject in need thereof. In some embodiments, the method comprises administering to the subject a dendrimer complexed, covalently conjugated, or intra-molecularly dispersed or encapsulated with one or more therapeutic or prophylactic agents, in an amount effective to treat, alleviate or prevent one or more symptoms of a kidney injury, disease or disorder. In some embodiments, the dendrimer is a. generation 4, 5, 6, 7, or 8 poly(amidoamine)(PAMAM) dendrimers, and the therapeutic agents are one or more anti-inflammatory agents and/or PPAR-δ agonists.

### CROSS-REFERENCE TO RELATED APPLICATIONS

This application claims priority under 35 U.S.C. § 119(e) to U.S. Provisional Patent Application No. 63/053,228, filed Jul. 17, 2020, which is hereby incorporated by reference in its entirety.

### BACKGROUND

The kidneys have a pivotal role in a number of basic physiological functions including blood pressure control, salt and water homeostasis, blood cell production, acid-base balance, and calcium homeostasis. Therefore, it is not surprising that renal dysfunction can result from, or cause, a variety of pathologies. Kidney diseases are typically classified as either chronic or acute. Whereas acute kidney injury (AKI) is commonly associated with bacterial infection, sepsis or ischemia-reperfusion injury (I/R that can transition to chronic renal disease), chronic kidney disease (CKD) typically results from diabetic complications, hypertension, obesity, and autoimmunity. The initiating events that promote renal disease can be quite different; however, AKI can lead to CKD, and, if unchecked, both can lead to end-stage renal disease (ESRD).

Glomerular and interstitial macrophage infiltration is a feature of both acute and chronic kidney diseases. Macrophages have been shown to be key players in renal injury, inflammation, and fibrosis. Macrophages are highly heterogeneous cells and exhibit distinct phenotypic and functional characteristics in response to various stimuli in the local microenvironment in different types of kidney disease. During renal inflammation, circulating monocytes are recruited and then become activated and polarized. By adapting to the local microenvironment, macrophages can differentiate into distinct phenotypes and function as a double-bladed sword in different stages of kidney disease. In general, M1 macrophages play a pathogenic role in boosting inflammatory renal injury, whereas M2 macrophages exert an anti-inflammatory and wound healing (or profibrotic) role during renal repair.

Currently, there are no drugs available for either preventing or treating AKI. The clinical manifestations are, in part, due to early-onset mitochondrial deficits that drive multiple pathophysiological events that lead to AKI and appear to be linked to the severity of AKI and progression to Chronic Kidney Disease (CKD).

Accordingly, in some aspects, the disclosure provides compositions and methods for reducing or preventing inflammation in the kidneys. In some aspects, the disclosure provides compositions that reduce or prevent the pathological processes associated with the development and progression of AKI and/or CKD, and methods of making and using thereof. In some aspects, the disclosure provides compositions and methods for selectively targeting active agents to pro-inflammatory cells at the site of inflammation in the kidneys associated with AKI and/or CKD.

### SUMMARY

In some aspects, the disclosure provides methods for treating or preventing one or more symptoms of a kidney injury, disease, and/or condition in a subject in need thereof. In some embodiments, the methods include administering to the subject dendrimers complexed, covalently conjugated, and/or intra-molecularly dispersed or encapsulated with one or more therapeutic or prophylactic agents, in an amount effective to treat, alleviate or prevent one or more symptoms of the kidney injury, disease, and/or condition.

In some embodiments, methods of treating a subject with acute kidney injury (AKI) and/or chronic kidney disease (CKD), in particular those caused by renal ischemia/reperfusion injury, include administering to the subject dendrimers complexed, covalently conjugated, or intra-molecularly dispersed or encapsulated with one or more therapeutic or prophylactic agents. In some embodiments, the dendrimers are administered in an amount effective to treat, alleviate or prevent one or more symptoms of AKI and/or CKD in the subject. In some embodiments, the AKI and/or CKD is associated with bacterial infection, sepsis, ischemia-reperfusion injury, diabetic complications, hypertension, obesity, and autoimmunity.

In some embodiments, the dendrimers are hydroxyl-terminated dendrimers. In some embodiments, a dendrimer is a hydroxyl-terminated dendrimer is a poly(amidoamine) (PAMAM) dendrimer, such as a generation 4, generation 5, or generation 6 poly(amidoamine) (PAMAM) dendrimer. In some embodiments, a therapeutic agent of the one or more therapeutic agents is a peroxisome proliferator-activated receptor delta (PPAR-δ) agonist. In some embodiments, a therapeutic agent of the one or more therapeutic agents is an anti-inflammatory agent. In some embodiments, the anti-inflammatory agent is N-acetyl cysteine.

In some embodiments, dendrimer compositions of the disclosure can be administered to reduce inflammation in the kidney, for example, to reduce tubular damage, tubular epithelial flattening, tubular dilatation, and tubular epithelial cell necrosis and/or apoptosis in the kidneys. In some embodiments, the dendrimer compositions are administered in an amount effective to reduce serum levels of creatinine and/or blood urea nitrogen (BUN); to reduce urine NGAL and/or KIM-1 content; and/or to improve glomerular filtration rate (GFR). In some embodiments, the methods comprise administering dendrimer compositions in an amount effective to reduce one or more pro-inflammatory cells, chemokines, and/or cytokines in the kidney, for example, to reduce one or more pro-inflammatory cytokines selected from TNF-α, IL-6, IL-12, IL-1β, and IL-18, or to reduce one or more pro-inflammatory cells such as M1-like macrophages.

In some aspects, the disclosure provides pharmaceutical compositions for use in treating or preventing one or more kidney injuries, diseases, and/or disorders in a subject in need thereof. In some embodiments, the dendrimer compositions can be stored or shipped as a dry powder and resuspended at the time of administration. In some embodiments, the dendrimer compositions are formulated for intravenous, subcutaneous, or intramuscular administration, and are administered via the intravenous, subcutaneous, or intramuscular route. Kits, including a container, containing one or more single unit dose of a composition including dendrimers covalently conjugated with one or more anti-inflammatory agents, and instructions on how the dose is to be administered for treatment of kidney injury are also described.

In some embodiments, the composition is administered prior to, in conjunction with, subsequent to, or in alternation with treatment with one or more additional therapies or procedures. In some embodiments, additional therapies or procedures include intravenous (i.v.) fluids in case of a lack of fluids in the blood, medications (e.g., diuretics) to induce expulsion of fluids (e.g., if too much fluid causes swelling in the limbs), medications to control blood potassium, such as calcium, glucose or sodium polystyrene sulfonate (KIONEX®), medications to restore blood calcium levels, such as an infusion of calcium, and/or hemodialysis to remove toxins in the body.

In some aspects, the disclosure provides a composition comprising a compound that comprises a dendrimer conjugated to a PPAR-δ agonist through an ester, ether, or amide linkage. In some embodiments, the dendrimer comprises a high density of surface hydroxyl groups. In some embodiments, the dendrimer is conjugated to the PPAR-S agonist through an ether or amide linkage. In some embodiments, the dendrimer is conjugated to the PPAR-S agonist through an ether linkage.

In some embodiments, the PPAR-δ agonist is conjugated to the ester, ether, or amide linkage through a spacer. In some embodiments, the spacer comprises alkyl groups, heteroalkyl groups, or alkylaryl groups. In some embodiments, the spacer comprises a peptide. In some embodiments, the spacer comprises polyethylene glycol.

In some embodiments, conjugation of the PPAR-δ agonist occurs on less than 50% of total available surface functional groups of the dendrimer prior to the conjugation. In some embodiments, conjugation of the PPAR-δ agonist occurs on less than occurs on less than 5%, less than 10%, less than 20%, less than 30%, or less than 40% of total available surface functional groups of the dendrimer prior to the conjugation.

In some embodiments, the PPAR-δ agonist is an indanylacetic acid derivative. In some embodiments, the PPAR-δ agonist is GW0742. In some embodiments, the PPAR-δ agonist is a GW0742-amide derivative or a GW0742-ester derivative.

In some embodiments, the dendrimer comprises poly(amidoamine), polypropylamine (POPAM), polyethylenimine, polylysine, polyester, iptycene, aliphatic poly(ether), and/or aromatic polyether. In some embodiments, the dendrimer is a poly(amidoamine) dendrimer. In some embodiments, the dendrimer is a generation 4, generation 5, or generation 6 poly(amidoamine) dendrimer.

In some embodiments, the zeta potential of the compound is between −25 mV and 25 mV. In some embodiments, the zeta potential of the compound is between −20 mV and 20 mV, between −10 mV and 10 mV, between −10 mV and 5 mV, between −5 mV and 5 mV, or between −2 mV and 2 mV. In some embodiments, the surface charge of the compound is neutral or near-neutral.

In some aspects, the disclosure provides use of the composition or the compound in treating one or more symptoms of a kidney injury, disease, and/or condition in a subject in need thereof. In some aspects, the disclosure provides use of the composition or the compound in the manufacture of a medicament for treating one or more symptoms of a kidney injury, disease, and/or condition in a subject in need thereof.

### BRIEF DESCRIPTION OF THE DRAWINGS

FIGS. 1A-1G are bar graphs showing levels of serum urea (FIG. 1A), serum creatinine (FIG. 1B), glomerular filtration rate (GFR) (FIG. 1C), concentrations of urine KIM-1 (FIG. 1D), concentrations of urine NGAL (FIG. 1E), quantity of KIM-1 in urine samples (FIG. 1F), and quantity of NGAL in urine samples (FIG. 1G) in four groups of experimental rats G1-G4 defined in Table 1. *p<0.05, **p<0.01, ***p<0.001, ****p<0.0001, compared to G1; #p<0.05, ##p<0.01, ####p<0.0001, compared to G2; &p<0.05, &&p<0.01, compared to G3.

FIGS. 2A-2C are bar graphs showing bilateral renal tubular degeneration score (FIG. 2A), bilateral renal tubular necrosis score (FIG. 2B), bilateral renal tubular total damage score (FIG. 2C) in the four groups of experimental rats G1-G4 defined in Table 1. **p<0.01, ***p<0.001, ****p<0.0001, compared to G1; #p<0.05, compared to G2.

FIGS. 3A-3B are bar graphs showing bilateral proximal tubular basement membrane damage (FIG. 3A) and bilateral proximal tubular brush border damage (FIG. 3B) in the four groups of experimental rats G1-G4 defined in Table 1. ****p<0.0001, compared to G1; ###p<0.001, compared to G2.

FIGS. 4A-4B are bar graphs showing renal tubular uptake of D-Cy5 (μm², FIG. 4A) and number of D-Cy5+ ED1+ cells (FIG. 4B) in the four groups of experimental rats G1-G4.

### DETAILED DESCRIPTION

Among other aspects, the disclosure provides dendrimer complexes (e.g., conjugates), compositions comprising dendrimer conjugates, and methods of using dendrimer conjugates and compositions thereof. In some embodiments, a dendrimer conjugate comprises a dendrimer conjugated to at least one agent. In some embodiments, a dendrimer conjugate comprises one or more agents useful in treating and/or diagnosing one or more symptoms of a kidney injury, disease, and/or condition.

In some aspects, the disclosure provides a compound comprising a dendrimer conjugated to a therapeutic agent. The inventors have recognized and appreciated that certain therapeutics with unfavorable in vivo profiles can be modified by conjugation to dendrimers having a high density of terminal hydroxyl groups (e.g., hydroxyl-terminated dendrimers) to provide a therapeutic compound that shows more highly selective uptake and increased localization to renal sites of inflammation. The inventors have further recognized and appreciated that such therapeutic compounds allow for the targeted delivery of certain therapeutics to biological targets in kidney which would otherwise be poorly accessible by the therapeutic.

### I. Compositions

In some aspects, the disclosure provides compositions of dendrimer complexes suitable for delivering one or more active agents, particularly one or more active agents to prevent, treat or diagnose a kidney injury, disease or disorder in a subject in need thereof. In some embodiments, the compositions are suitable for treating acute kidney injury (AKI) and chronic kidney disease (CKD) caused by ischemia/reperfusion injury (IRI).

Compositions of dendrimer complexes including one or more prophylactic, therapeutic, and/or diagnostic agents encapsulated, associated, and/or conjugated in the dendrimers are provided. Generally, in some embodiments, one or more active agent is encapsulated, associated, and/or conjugated in the dendrimer complex at a concentration of about 0.01% to about 30%, about 1% to about 20%, or about 5% to about 20% by weight. In some embodiments, an active agent is covalently conjugated to the dendrimer via one or more linkages such as disulfide, ester, ether, thioester, carbamate, carbonate, hydrazine, and amide, optionally via one or more spacers. In some embodiments, the spacer is an active agent, such as N-acetyl cysteine. Exemplary active agents include anti-inflammatory drugs and PPAR-δ agonists.

The presence of the additional agents can affect the zeta-potential or the surface charge of the particle. In one embodiment, the zeta potential of the dendrimers is between −100 mV and 100 mV, between −50 mV and 50 mV, between −25 mV and 25 mV, between −20 mV and 20 mV, between −10 mV and 10 mV, between −10 mV and 5 mV, between −5 mV and 5 mV, or between −2 mV and 2 mV. In some embodiments, the surface charge is neutral or near-neutral. The range above is inclusive of all values from −100 mV to 100 mV.

Dendrimers

Dendrimers are three-dimensional, hyperbranched, monodispersed, globular and polyvalent macromolecules including a high density of surface end groups (Tomalia, D. A., el al., Biochemical Society Transactions, 35, 61 (2007); and Sharma, A., e al., ACS Macro Letters, 3, 1079 (2014)). Due to their unique structural and physical features, dendrimers are useful as nano-carriers for various biomedical applications including targeted drug/gene delivery, imaging and diagnosis (Sharma, A., et al., RSC Advances, 4, 19242 (2014); Caminade, A.-M., et al., Journal of Materials Chemistry B, 2, 4055 (2014); Esfand, R., et al., Drug Discovery Today, 6, 427 (2001); and Kannan, R M., et al., Journal of Internal Medicine, 276, 579 (2014)).

Dendrimer surface groups have a significant impact on their biodistribution (Nance, E., et al., Biomaterials, 101, 96 (2016)). Hydroxyl terminated generation 4 PAMAM dendrimers (˜4 nm size) without any targeting ligand cross the impaired BBB upon systemic administration in a rabbit model of cerebral palsy (CP) significantly more (>20 fold) as compared to healthy controls, and selectively target activated microglia and astrocytes (Lesniak, W. G., et al., Mol Pharm, 10 (2013)).

The term “dendrimer” includes, but is not limited to, a molecular architecture with an interior core and layers (or “generations”) of repeating units which are attached to and extend from this interior core, each layer having one or more branching points, and an exterior surface of terminal groups attached to the outermost generation. In some embodiments, dendrimers have regular dendrimeric or “starburst” molecular structures.

In some embodiments, dendrimers have a diameter of between about 1 nm and about 50 nm, between about 1 nm and about 20 nm, between about 1 nm and about 10 nm, or between about 1 nm and about 5 nm. In some embodiments, the diameter is between about 1 nm and about 2 nm. Conjugates are generally in the same size range, although large proteins such as antibodies may increase the size by 5-15 nm. In some embodiments, agent is encapsulated in a ratio of agent to dendrimer of between 1:1 and 4:1 for the larger generation dendrimers. In some embodiments, the dendrimers have a diameter effective to penetrate kidney epithelial tissue and to retain in target cells for a prolonged period.

In some embodiments, dendrimers have a molecular weight of between about 500 Daltons and about 100,000 Daltons, between about 500 Daltons and about 50,000 Daltons, or between about 1,000 Daltons and about 20,000 Daltons.

Suitable dendrimer scaffolds that can be used include poly(amidoamine), also known as PAMAM, or STARBURST™ dendrimers; polypropylamine (POPAM), polyethylenimine, polylysine, polyester, iptycene, aliphatic poly(ether), and/or aromatic polyether dendrimers. The dendrimers can have carboxylic, amine and/or hydroxyl terminations. In some embodiments, the dendrimers have hydroxyl terminations (e.g., a hydroxyl-terminated dendrimer). Each dendrimer of the dendrimer complex may be of the same or of similar or different chemical nature than the other dendrimers (e.g., the first dendrimer may include a PAMAM dendrimer, while the second dendrimer may be a POPAM dendrimer).

In some embodiments, the term “PAMAM dendrimer” means a poly(amidoamine) dendrimer, which may contain different cores, with amidoamine building blocks, and can have carboxylic, amine and hydroxyl terminations of any generation including, but not limited to, generation 1 PAMAM dendrimers, generation 2 PAMAM dendrimers, generation 3 PAMAM dendrimers, generation 4 PAMAM dendrimers, generation 5 PAMAM dendrimers, generation 6 PAMAM dendrimers, generation 7 PAMAM dendrimers, generation 8 PAMAM dendrimers, generation 9 PAMAM dendrimers, or generation 10 PAMAM dendrimers. In some embodiments, the dendrimers are soluble in the formulation and are generation (“G”) 4, 5 or 6 dendrimers (i.e., G4-G6 dendrimers), and/or G4-G10 dendrimers, G6-G10 dendrimers, or G2-G10 dendrimers. The dendrimers may have hydroxyl groups attached to their functional surface groups.

Methods for making dendrimers are known to those of skill in the art and generally involve a two-step iterative reaction sequence that produces concentric shells (generations) of dendritic β-alanine units around a central initiator core (e.g., ethylenediamine-cores). Each subsequent growth step represents a new “generation” of polymer with a larger molecular diameter, twice the number of reactive surface sites, and approximately double the molecular weight of the preceding generation. Dendrimer scaffolds suitable for use are commercially available in a variety of generations. In some embodiments, the dendrimer compositions are based on generation 0, 1, 2, 3, 4, 5, 6, 7, 8, 9, or 10 dendrimeric scaffolds. Such scaffolds have, respectively, 4, 8, 16, 32, 64, 128, 256, 512, 1024, 2048, and 4096 reactive sites. Thus, the dendrimeric compounds based on these scaffolds can have up to the corresponding number of agents or moities bound thereto, directly or indirectly through a linker.

In some embodiments, the dendrimers include a plurality of hydroxyl groups. Some exemplary high-density hydroxyl group-containing dendrimers include commercially available polyester dendritic polymer such as hyperbranched 2,2-Bis(hydroxyl-methyl)propionic acid polyester polymer (for example, hyperbranched bis-MPA polyester-64-hydroxyl, generation 4), dendritic polyglycerols.

In some embodiments, the high-density hydroxyl group-containing dendrimers are oligo ethylene glycol (OEG)-like dendrimers. For example, a generation 2 OEG dendrimer (D2-OH-60) can be synthesized using highly efficient, robust and atom economical chemical reactions such as Cu (I) catalyzed alkyne-azide click and photo catalyzed thiol-ene click chemistry. Highly dense polyol dendrimer at very low generation in minimum reaction steps can be achieved by using an orthogonal hypermonomer and hypercore strategy, for example as described in International Patent Publication No. WO2019094952. In some embodiments, the dendrimer backbone has non-cleavable polyether bonds throughout the structure to avoid the disintegration of dendrimer in vivo and to allow the elimination of such dendrimers as a single entity from the body (non-biodegradable).

In some embodiments, the dendrimer specifically targets a particular tissue region and/or cell type, pro-inflammatory macrophages involved in ALI/ARDS. In some embodiments, the dendrimer specifically targets a particular tissue region and/or cell type without a targeting moiety.

In some embodiments, the dendrimers have a plurality of hydroxyl (—OH) groups on the periphery of the dendrimers. In some embodiments, the surface density of hydroxyl (—OH) groups is at least 1 OH group/nm² (number of hydroxyl surface groups/surface area in nm²). For example, in some embodiments, the surface density of hydroxyl groups is more than 2, 3, 4, 5, 6, 7, 8, 9, or 10 groups/nm²; at least 10, 15, 20, 25, 30, 35, 40, 45, 50, or more than 50 groups/nm². In further embodiments, the surface density of hydroxyl (—OH) groups is between about 1 and about 50, e.g., 5-20 OH groups/nm² (number of hydroxyl surface groups/surface area in nm²), while having a molecular weight of between about 500 Da and about 10 kDa.

In some embodiments, the dendrimers may have a fraction of the hydroxyl groups exposed on the outer surface, with the others in the interior core of the dendrimers. In some embodiments, the dendrimers have a volumetric density of hydroxyl (—OH) groups of at least 1 OH group/nm³ (number of hydroxyl groups/volume in nm³). For example, in some embodiments, the volumetric density of hydroxyl groups is 2, 3, 4, 5, 6, 7, 8, 9, 10, or more than 10, 15, 20, 25, 30, 35, 40, 45, and 50 groups/nm³. In some embodiments, the volumetric density of hydroxyl groups is between about 4 and about 50 groups/nm³, between about 5 and about 30 groups/nm³, or between about 10 and about 20 groups/nm³.

Dendrimers can be purchased or prepared via a variety of chemical reaction steps. Dendrimers are usually synthesized according to methods allowing controlling their structure at every stage of construction. The dendritic structures are mostly synthesized by two main different approaches: divergent or convergent.

In some embodiments, dendrimers are prepared using divergent methods, in which the dendrimer is assembled from a multifunctional core, which is extended outward by a series of reactions, commonly a Michael reaction. The strategy involves the coupling of monomeric molecules that possesses reactive and protective groups with the multifunctional core moiety, which leads to stepwise addition of generations around the core followed by removal of protecting groups. For example, PAMAM-NH₂ dendrimers are first synthesized by coupling N-(2-aminoethyl) acryl amide monomers to an ammonia core.

In other embodiments, dendrimers are prepared using convergent methods, in which dendrimers are built from small molecules that end up at the surface of the sphere, and reactions proceed inward building inward and are eventually attached to a core.

Many other synthetic pathways exist for the preparation of dendrimers, such as the orthogonal approach, accelerated approaches, the Double-stage convergent method or the hypercore approach, the hypermonomer method or the branched monomer approach, the Double exponential method; the Orthogonal coupling method or the two-step approach, the two monomers approach, AB₂-CD₂ approach.

In some embodiments, the core of the dendrimer, one or more branching units, one or more linkers/spacers, and/or one or more surface groups can be modified to allow conjugation to further functional groups (branching units, linkers/spacers, surface groups, etc.), monomers, and/or active agents via click chemistry, employing one or more Copper-Assisted Azide-Alkyne Cycloaddition (CuAAC), Diels-Alder reaction, thiol-ene and thiol-yne reactions, and azide-alkyne reactions (Arseneault M et al., Molecules. 2015 May 20; 20(5):9263-94). In some embodiments, pre-made dendrons are clicked onto high-density hydroxyl polymers. ‘Click chemistry’ involves, for example, the coupling of two different moieties (e.g., a core group and a branching unit; or a branching unit and a surface group) via a 1,3-dipolar cycloaddition reaction between an alkyne moiety (or equivalent thereof) on the surface of the first moiety and an azide moiety (e.g., present on a triazine composition or equivalent thereof), or any active end group such as, for example, a primary amine end group, a hydroxyl end group, a carboxylic acid end group, a thiol end group, etc.) on the second moiety.

In some embodiments, dendrimer synthesis replies upon one or more reactions such as thiol-ene click reactions, thiol-yne click reactions, CuAAC, Diels-Alder click reactions, azide-alkyne click reactions, Michael Addition, epoxy opening, esterification, silane chemistry, and a combination thereof.

Any existing dendritic platforms can be used to make dendrimers of desired functionalities, i.e., with a high-density of surface hydroxyl groups by conjugating high-hydroxyl containing moieties such as 1-thio-glycerol or pentaerythritol. Exemplary dendritic platforms such as polyamidoamine (PAMAM), poly (propylene imine) (PPI), poly-L-lysine, melamine, poly (etherhydroxylamine) (PEHAM), poly (esteramine) (PEA) and polyglycerol can be synthesized and explored.

Dendrimers also can be prepared by combining two or more dendrons. Dendrons are wedge-shaped sections of dendrimers with reactive focal point functional groups. Many dendron scaffolds are commercially available. They come in 1, 2, 3, 4, 5, and 6th generations with, respectively, 2, 4, 8, 16, 32, and 64 reactive groups. In certain embodiments, one type of active agents are linked to one type of dendron and a different type of active agent is linked to another type of dendron. The two dendrons are then connected to form a dendrimer. The two dendrons can be linked via click chemistry i.e., a 1,3-dipolar cycloaddition reaction between an azide moiety on one dendron and alkyne moiety on another to form a triazole linker.

Exemplary methods of making dendrimers are described in detail in International Patent Publication Nos. WO2009/046446, WO2015168347, WO2016025745, WO2016025741, WO2019094952, and U.S. Pat. No. 8,889,101.

Dendrimer Complexes/Conjugates

Dendrimer complexes can be formed of therapeutic, prophylactic or diagnostic agents conjugated or complexed to a dendrimer, a dendritic polymer or a hyperbranched polymer. Conjugation of one or more agents to a dendrimer are known in the art, and are described in detail in U.S. Published Application Nos. US 2011/0034422, US 2012/0003155, and US 2013/0136697.

In some embodiments, one or more active agents are covalently attached to the dendrimers. In some embodiments, the active agents are functionalized for conjugation to the dendrimer, optionally via one or more linking moieties. The functionalized active agents and/or linking moieties are designed to have desirable release rate of the active agents from the dendrimers in vivo. The functionalized active agents and/or linking moieties can be designed to be cleaved hydrolytically, enzymatically, or combinations thereof, to provide for the sustained release of the active agents in vivo. In the case where cleavable forms are desired, both the composition of the linking moiety and its point of attachment to the active agent, are selected so that cleavage of the linking moiety releases either an active agent, or a suitable prodrug thereof. In some embodiments, the functionalized active agents and/or linking moieties are designed to be cleaved at a minimal or insignificant rate in vivo. The composition of the linking moiety can also be selected in view of the desired release rate of the active agents. In some embodiments, one or more active agents are functionalized to be non-cleavable or minimally cleavable from the dendrimers in vivo, for example via ether linkage, optionally, with one or more spacers/linkers.

In some embodiments, the attachment occurs via one or more of disulfide, ester, ether, thioester, carbamate, carbonate, hydrazine, or amide linkages. In some embodiments, the attachment occurs via an appropriate spacer that provides an ester bond or an amide bond between the agent and the dendrimer depending on the desired release kinetics of the active agent. In some cases, an ester bond is introduced for releasable form of active agents. In other cases, an amide bond is introduced for non-releasable form of active agents.

Linking moieties can include one or more organic functional groups. Examples of suitable organic functional groups include secondary amides (—CONH—), tertiary amides (—CONR—), sulfonamide (—S(O)₂—NR—), secondary carbamates (—OCONH—; —NHCOO—), tertiary carbamates (—OCONR—; —NRCOO—), carbonate (—O—C(O)—O—), ureas (—NHCONH—; —NRCONH—; —NHCONR—, —NRCONR—), carbinols (—CHOH—, —CROH—), disulfide groups, hydrazones, hydrazides, ethers (—O—), and esters (—COO—, —CH₂O₂C—, CHRO₂C—), wherein R is an alkyl group, an aryl group, or a heterocyclic group. In general, the identity of the one or more organic functional groups within the linking moiety can be chosen in view of the desired release rate of the active agents. In addition, the one or more organic functional groups can be chosen to facilitate the covalent attachment of the active agents to the dendrimers. In some embodiments, the attachment can occur via an appropriate spacer that provides a disulfide bridge between the agent and the dendrimer. In some embodiments, the dendrimer complexes are capable of rapid release of the agent in vivo by thiol exchange reactions, under the reduced conditions found in body.

In certain embodiments, the linking moiety includes one or more of the organic functional groups described above in combination with a spacer group. The spacer group can be composed of any assembly of atoms, including oligomeric and polymeric chains; for example, in some embodiments, the total number of atoms in the spacer group is between 3 and 200 atoms, between 3 and 150 atoms, between 3 and 100 atoms, or between 3 and 50 atoms. Examples of suitable spacer groups include alkyl groups, heteroalkyl groups, alkylaryl groups, oligo- and polyethylene glycol chains, and oligo- and poly(amino acid) chains. Variation of the spacer group provides additional control over the release of the agents in vivo. In embodiments where the linking moiety includes a spacer group, one or more organic functional groups will generally be used to connect the spacer group to both the active agent and the dendrimers.

Reactions and strategies useful for the covalent attachment of agents to dendrimers are known in the art. See, for example, March, “Advanced Organic Chemistry,” 5th Edition, 2001, Wiley-Interscience Publication, New York) and Hermanson, “Bioconjugate Techniques,” 1996, Elsevier Academic Press, U.S.A. Appropriate methods for the covalent attachment of a given active agent can be selected in view of the linking moiety desired, as well as the structure of the agents and dendrimers as a whole as it relates to compatibility of functional groups, protecting group strategies, and the presence of labile bonds.

The optimal drug loading will necessarily depend on many factors, including the choice of drug, dendrimer structure and size, and tissues to be treated. In some embodiments, the one or more active drugs are encapsulated, associated, and/or conjugated to the dendrimer at a concentration of about 0.01% to about 45%, about 0.1% to about 30%, about 0.1% to about 20%, about 0.1% to about 10%, about 1% to about 10%, about 1% to about 5%, about 3% to about 20% by weight, and about 3% to about 10% by weight. However, optimal drug loading for any given drug, dendrimer, and site of target can be identified by routine methods, such as those described.

In some embodiments, conjugation of active agents and/or linkers occurs through one or more surface and/or interior groups. Thus, in some embodiments, the conjugation of active agents/linkers occurs via about 1%, 2%, 3%, 4%, or 5% of the total available surface functional groups, such as hydroxyl groups, of the dendrimers prior to the conjugation. In other embodiments, the conjugation of active agents/linkers occurs on less than 5%, less than 10%, less than 15%, less than 20%, less than 25%, less than 30%, less than 35%, less than 40%, less than 45%, less than 50%, less than 55%, less than 60%, less than 65%, less than 70%, less than 75% total available surface functional groups of the dendrimers prior to the conjugation. In some embodiments, dendrimer complexes retain an effective amount of surface functional groups for targeting to specific cell types, whilst conjugated to an effective amount of active agents for treat, prevent, and/or image the disease or disorder.

In some aspects, the disclosure provides therapeutic and/or diagnostic compounds comprising a dendrimer conjugated to an agent through a terminal ester, ether, or amide bond. In some embodiments, the dendrimer comprises surface (e.g., terminal) hydroxyl groups optionally substituted with the agent. In some embodiments, the agent is a therapeutic agent or a diagnostic agent (e.g., an imaging agent).

In some aspects, the disclosure provides a composition comprising a therapeutic compound that comprises a dendrimer conjugated to a therapeutic agent through a terminal ester, ether, or amide bond. In some embodiments, the dendrimer comprises a high-density of terminal hydroxyl groups optionally substituted with the therapeutic agent. In some embodiments, a therapeutic compound comprising a dendrimer conjugated to a therapeutic agent is 10-20% by mass of therapeutic agent. In some embodiments, the terminal ester, ether, or amide bond is conjugated to the therapeutic agent through a linker.

In some embodiments, the therapeutic compound is about 10% to about 15% by mass of therapeutic agent. In some embodiments, the therapeutic compound is about 15% to about 20% by mass of therapeutic agent. In some embodiments, at least 50% of terminal sites on the dendrimer comprise terminal hydroxyl groups. In some embodiments, at least 50% and up to 99% (e.g., 50-95%, 50-90%, 50-80%, 50-70%, 50-60%, 60-80%, 70-90%) of terminal sites on the dendrimer comprise terminal hydroxyl groups.

In some embodiments, the therapeutic agent has an aqueous solubility that is increased relative to an unconjugated compound comprising the therapeutic agent in absence of the dendrimer. In some embodiments, the aqueous solubility is increased by at least 10% relative to the unconjugated compound. In some embodiments, the aqueous solubility is increased by between about 10% and about 100% relative to the unconjugated compound. In some embodiments, the aqueous solubility is increased by at least about a factor of two relative to the unconjugated compound. In some embodiments, the aqueous solubility is increased by between about a factor of two and about a factor of ten relative to the unconjugated compound. In some embodiments, the aqueous solubility is solubility under physiological conditions. In some embodiments, the aqueous solubility is solubility in water having a pH of between about 7.0 and about 8.0. In some embodiments, the therapeutic agent is present at a concentration at which the unconjugated compound is insoluble under physiological conditions.

In some embodiments, surface functional groups (e.g., terminal functional groups) of a dendrimer include one or more hydroxyl groups, one or more amine groups, and/or one or more carboxyl groups. In some embodiments, the terminal functional groups of a dendrimer provide attachment sites through which the at least one agent is conjugated to form the dendrimer conjugate. Accordingly, in some embodiments, the at least one agent is conjugated to the dendrimer through an ether bond, an amide bond, or an ester bond formed by conjugation to a terminal functional group of the dendrimer. In some embodiments, the at least one agent is conjugated to the dendrimer through an ether bond or an amide bond. In some embodiments, the at least one agent is conjugated to the dendrimer through an ether bond.

In some embodiments, the number of terminal sites on a dendrimer can depend on the particular dendrimeric scaffold and its generation. For example, in some embodiments, a dendrimer is based on a generation 0, 1, 2, 3, 4, 5, 6, 7, 8, 9, or 10 PAMAM dendrimeric scaffold, which have 4, 8, 16, 32, 64, 128, 256, 512, 1024, 2048, and 4096 terminal sites, respectively. However, it should be appreciated that different dendrimeric scaffolds having a different number of terminal sites at each generation can be used in accordance with the disclosure.

In some embodiments, all terminal sites of a dendrimer comprise hydroxyl groups. In some embodiments, each terminal site of a dendrimer comprises either a hydroxyl group or an amine group. In some embodiments, each terminal site of a dendrimer conjugate comprises a hydroxyl group, an amine group, or an agent conjugated to the dendrimer through an ether or amide bond. In some embodiments, each terminal site of a dendrimer conjugate comprises either a hydroxyl group or an agent conjugated to the dendrimer through an ether bond.

In some embodiments, at least 50% of terminal sites on a dendrimer conjugate comprise hydroxyl groups (e.g., at least 50% of terminal sites do not comprise either an amine group or an agent). For example, in some embodiments, at least 60%, at least 70%, at least 80%, at least 90%, at least 95%, at least 98%, or at least 99% of terminal sites on a dendrimer conjugate comprise hydroxyl groups. In some embodiments, about 50-99%, about 60-99%, about 70-99%, about 80-99%, about 90-99%, about 95-99%, about 98-99%, about 70-95%, about 70-90%, about 80-95%, or about 80-90% of terminal sites on a dendrimer conjugate comprise hydroxyl groups.

In some embodiments, one or more terminal sites on a dendrimer conjugate comprise an agent. In some embodiments, at least 2, at least 3, at least 4, at least 5, at least 10, at least 15, at least 20, or more, terminal sites on a dendrimer conjugate comprise an agent. In some embodiments, at least 1% of terminal sites on a dendrimer conjugate comprise an agent. For example, in some embodiments, at least 2%, at least 5%, at least 10%, at least 15%, at least 20%, at least 25%, or at least 30% of terminal sites on a dendrimer conjugate comprise an agent. In some embodiments, about 1-50%, about 1-40%, about 1-25%, about 1-10%, about 5-50%, about 5-40%, about 5-25%, about 5-10%, about 10-50%, about 10-40%, or about 10-25% of terminal sites on a dendrimer conjugate comprise an agent. In some embodiments, about 1%, about 2%, about 3%, about 4%, or about 5% of terminal sites on a dendrimer comprise an agent. In some embodiments, less than 5%, less than 10%, less than 15%, less than 20%, less than 25%, less than 30%, less than 35%, less than 400%, less than 45%, less than 50%, less than 55%, less than 60%, less than 65%, less than 70%, less than 75% of terminal sites on a dendrimer comprise an agent. In some embodiments, a dendrimer conjugate has an effective amount of terminal functional groups (e.g., terminal hydroxyl groups) for targeting to a specific cell type, while having to an effective amount of agent for treating and/or imaging as described herein. In some embodiments, terminal sites of a dendrimer conjugate can be evaluated using proton nuclear magnetic resonance (¹H NMR), or other analytical methods known in the art, to determine a percentage of terminal sites having an agent and/or terminal functional group.

In some embodiments, a desired agent loading can depend on certain factors, including the choice of agent, dendrimer structure and size, and cell or tissue to be treated. In some embodiments, a dendrimer conjugate (e.g., a therapeutic compound) is about 0.01% to about 45% by mass (m/m) of agent (e.g., therapeutic agent). In some embodiments, a dendrimer conjugate (e.g., a therapeutic compound) is about 10% to about 20% by mass of agent (e.g., therapeutic agent). In some embodiments, a dendrimer conjugate is about 0.1% to about 30%, about 0.1% to about 20%, about 0.1% to about 10%, about 1% to about 10%, about 1% to about 5%, about 3% to about 20%, about 3% to about 10% by mass of agent.

As described herein, in some embodiments, a dendrimer conjugate can be characterized in terms of mass percentage (e.g., % by mass (m/m)) of agent. In some embodiments, mass percentage refers to a molecular weight (Da) percentage of agent in a dendrimer conjugate. In some embodiments, mass percentage can be determined by the general formula of: (agent M_{W})/(conjugate M_{W})×100. For example, in some embodiments, (agent M_{W}) can be determined by calculating or approximating the molecular weight of an agent as a single molecule or compound (conjugated or unconjugated), and multiplying this value by the number of terminal sites at which the agent is present in a dendrimer conjugate. In some embodiments, (agent M_{W}) can be determined by calculating or approximating the sum of the atomic mass of all atoms which form the agent in a dendrimer conjugate. The value for (agent M_{W}) can be taken as a fraction of total molecular weight of the dendrimer conjugate (conjugate M_{W}), and multiplied by 100 to provide a mass percentage. In some embodiments, mass percentage can be determined by experimental or empirical means. For example, in some embodiments, mass percentage can be determined using proton nuclear magnetic resonance (¹H NMR) or other analytical methods known in the art.

In some embodiments, a dendrimer has a diameter of between about 1 nm and about 50 nm. For example, in some embodiments, the diameter is between about 1 nm and about 20 nm, between about 1 nm and about 10 nm, or between about 1 nm and about 5 nm. In some embodiments, the diameter is between about 1 nm and about 2 nm. In some embodiments, a dendrimer that is conjugated to a relatively large agent (e.g., a large protein, such as an antibody) can have a diameter that increases these values by approximately 5-15 nm relative to the unconjugated dendrimer. In some embodiments, a dendrimer has a molecular weight of between about 500 Daltons (Da) and about 100,000 Da (e.g., between about 500 Da and about 50,000 Da, or between about 1,000 Da and about 20,000 Da).

In some embodiments, a dendrimer of a conjugate described herein is a poly(amidoamine) (PAMAM) dendrimer, a polypropylamine (POPAM) dendrimer, a 2,2-bis(hydroxymethyl)propionic acid (bis-MPA) dendrimer, a polyethylenimine dendrimer, a polylysine dendrimer, a polyester dendrimer, an iptycene dendrimer, aliphatic poly(ether) dendrimer, an aromatic polyether dendrimer, or a combination thereof.

In some embodiments, a dendrimer conjugate comprises a PAMAM dendrimer. In some embodiments, a PAMAM dendrimer comprises different cores with amidoamine building blocks. In some embodiments, a PAMAM dendrimer comprises carboxylic, amine, and/or hydroxyl terminal groups of any generation including, but not limited to, generation 1, generation 2, generation 3, generation 4, generation 5, generation 6, generation 7, generation 8, generation 9, or generation 10 PAMAM dendrimers. In some embodiments, a PAMAM dendrimer is a generation 4, generation 5, generation 6, generation 7, or generation 8 hydroxyl-terminated PAMAM dendrimer.

In some embodiments, the dendrimers include a plurality of hydroxyl groups. Some exemplary high-density hydroxyl groups-containing dendrimers include commercially available polyester dendritic polymer such as hyperbranched 2,2-Bis(hydroxyl-methyl)propionic acid polyester polymer (for example, hyperbranched bis-MPA polyester-64-hydroxyl, generation 4), dendritic polyglycerols. In some embodiments, the high-density hydroxyl groups-containing dendrimers are oligo ethylene glycol (OEG)-like dendrimers. For example, a generation 2 OEG dendrimer (D2-OH-60) can be synthesized using highly efficient, robust and atom economical chemical reactions such as Cu (I) catalyzed alkyne-azide click and photo catalyzed thiol-ene click chemistry. Highly dense polyol dendrimer at very low generation in minimum reaction steps can be achieved by using an orthogonal hypermonomer and hypercore strategy, for example as described in WO 2019094952. In some embodiments, dendrimer backbone has non-cleavable polyether bonds throughout the structure to avoid the disintegration of dendrimer in vivo, and to allow the elimination of such dendrimers as a single entity from the body (e.g., non-biodegradable).

In some embodiments, a dendrimer conjugate comprises a dendrimer that is conjugated to one or more therapeutic agents, one or more imaging agents, and/or one or more targeting agents. It should be appreciated that, in some embodiments, “at least one” agent, “one or more” agents, and similar terminology refer to a particular agent and not necessarily the amount of the particular agent that is conjugated to a dendrimer. For example, in some embodiments, a dendrimer conjugate comprising two agents refers to a dendrimer having a first agent at one or more terminal positions and a second agent at one or more different terminal positions, where the first and second agents are different (e.g., chemically different). In some embodiments, the first and second agents may be useful for a similar purpose (e.g., both agents are therapeutic agents), or the first and second agents may be useful for different purposes (e.g., the first agent is a therapeutic agent, and the second agent is a targeting agent). When used for a similar purpose, the first and second agents are chemically different and can therefore provide different functionalities—for example, different therapeutic agents targeting different receptors or biological pathways, or different imaging agents having different spectral properties.

In some embodiments, an agent (e.g., a therapeutic agent, an imaging agent, a targeting agent) of a dendrimer conjugate is a peptide, a protein, a sugar, a carbohydrate, an oligonucleotide, a nucleic acid, a lipid, a small-molecule compound, or a combination thereof. In some embodiments, an agent is an antibody or an antigen-binding fragment of an antibody. In some embodiments, an agent is a nucleic acid or oligonucleotide that encodes a protein, such as a DNA expression vector or an mRNA. In some embodiments, an agent is an RNA-silencing agent, such as an siRNA, shRNA, or a microRNA.

In some embodiments, an agent is a small-molecule compound, such as a small-molecule organic, organometallic, or inorganic compound. In some embodiments, an agent is a small-molecule compound having a molecular weight of less than 2,000 daltons (Da), less than 1,500 Da, less than 1,000 Da, or less than 500 Da. In some embodiments, an agent is a small-molecule compound having a molecular weight of between about 100 and about 2,000 Da. For example, in some embodiments, the small-molecule compound has a molecular weight of between about 100 and about 1,500 Da, between about 100 and about 1,000 Da, between about 500 and about 2,000 Da, or between about 300 and about 700 Da.

In some aspects, a non-releasable form of a dendrimer conjugate described herein provides enhanced therapeutic efficacy as compared to a releasable form of the same conjugate. Accordingly, in some embodiments, an agent is conjugated to a dendrimer through a linker, which is attached to the dendrimer and to the agent in a non-releasable manner (e.g., by ether and/or amide bonds). In some embodiments, a linker has a composition that is minimally releasable (e.g., minimally cleavable) under physiological conditions.

In some embodiments, a dendrimer is conjugated to an agent through covalent bonds that are stable under in vivo conditions. In some embodiments, the covalent bonds are minimally cleavable when administered to a subject and/or excreted intact from the body. For example, in some embodiments, less than 10%, 5%, 4%, 3%, 2%, 1%, 0.5%, 0.4%, 0.3%, 0.2%, 0.1%, or less than 0.1% of the total dendrimer conjugates have agent cleaved within 24 hours, or 48 hours, or 72 hours after in vivo administration to a subject. In some embodiments, the covalent bonds comprise ether bonds. In some embodiments, the covalent bonds between dendrimer and agent are not hydrolytically or enzymatically cleavable bonds, such as ester bonds.

In some aspects, the disclosure provides a dendrimer conjugate of Formula (I):

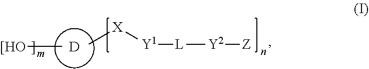

wherein: D is a dendrimer; X is O or NH; Y¹ is a first group; Y² is a second group; Z is an agent; L is a linker; m is an integer from 16 to 4096, inclusive; and n is an integer from 1 to 100, inclusive.

In some embodiments, D is a dendrimer selected from the group consisting of poly(amidoamine) (PAMAM) polymers, polypropylamine (POPAM) polymers, polyethylenimine polymers, polylysine polymers, polyester polymers, iptycene polymers, aliphatic poly(ether) polymers, aromatic polyether polymers, 2,2-bis(hydroxymethyl)propionic acid (bis-MPA) polymers, and combinations thereof.

In some embodiments, Y¹ is non-hydrolyzable under physiological conditions. In some embodiments, Y¹ is optionally substituted alkylene, optionally substituted alkenylene, optionally substituted alkynylene, or a covalent bond. In some embodiments, Y¹ is optionally substituted C₁-C₂₀ alkylene. In some embodiments, Y¹ is unsubstituted C₁-C₁₀ alkylene.

In some embodiments, Y² is selected from the group consisting of secondary amides, tertiary amides, sulfonamide, secondary carbamates, tertiary carbamates, carbonates, ureas, carbinols, disulfides, hydrazones, hydrazides, ethers, carbonyls, and combinations thereof. In some embodiments, Y² is selected from the group consisting of —CONH—, —CONR^{A}—, —SO₂NR^{A}—, —OCONH—, —NHCOO—, —OCONR^{A}—, —NR^{A}COO—, —OC(═O)O—, —NHCONH—, —NR^{A}CONH—, —NHCONR^{A}—, —NRCONR^{A}—, —CHOH—, —CR^{A}OH—, —C(═O)—, and —C(═O)R^{A}—, wherein R^{A} is an optionally substituted alkyl group, an optionally substituted aryl group, or an optionally substituted heterocyclic group.

In some embodiments, Z is a therapeutic agent, an imaging agent, or a targeting agent as described herein. In some embodiments, Z is a therapeutic agent or an imaging agent. In some embodiments, the dendrimer conjugate of Formula (I) further comprises at least one targeting agent conjugated to the dendrimer. In some embodiments, at least one of Z is a PPAR agonist (e g., a PPAR-δ agonist).

In some embodiments, L is a linker comprising a polymer and at least one moiety. In some embodiments, the polymer is a polymeric polyol, a polypeptide, or an unsubstituted alkyl chain. In some embodiments, the polymer is a polymeric polyol selected from the group consisting of polyethylene glycol (PEG), polypropylene glycol, and polyvinyl alcohol. In some embodiments, the polymer is a polypeptide having at least 2 amino acids. In some embodiments, the polymer is a polypeptide having between about 2 and about 40 amino acids (e.g., 2-25, 5-30, 10-25, or 5-15 amino acids). In some embodiments, the polymer is an unsubstituted alkyl chain. In some embodiments, the polymer is an unsubstituted C₂₋₅₀ alkyl chain. In some embodiments, the polymer is an unsubstituted C₂₋₃₀ alkyl chain. In some embodiments, the polymer is an unsubstituted C₅₋₂₅ alkyl chain. In some embodiments, the polymer is a polymer as described elsewhere herein.

In some embodiments, the at least one moiety of L is a moiety resulting from a click reaction. In some embodiments, the at least one moiety is a 5-membered heterocyclic ring resulting from an electrocyclic reaction (e.g., 3+2 cycloaddition, or 4+2 cycloaddition) between reactive click chemistry handles (e.g., azides and terminal or strained alkynes, dienes and dienophiles, thiols and alkenes) used to produce the conjugate. In some embodiments, the at least one moiety is a diradical comprising 1,2,3-triazolyl, 4,5-dihydro-1,2,3-triazolyl, isoxazolyl, 4,5-dihydroisoxazolyl, or 1,4-dihydropyridazyl.

In some aspects, the disclosure provides a dendrimer conjugate of Formula (II):

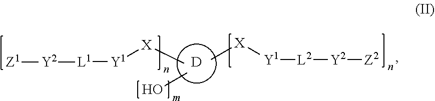

wherein: D, m, each instance of n, each instance of X, each instance of Y¹, and each instance of Y² is independently as defined with respect to Formula (I); L¹ and L² are independently linkers as defined with respect to Formula (I); and Z¹ and Z² are different agents.

In some embodiments, Z¹ and Z² are independently therapeutic agents, targeting agents, or imaging agents, with the proviso that Z¹ and Z² are different (e.g., chemically different). In some embodiments, Z¹ and Z² are different therapeutic agents. In some embodiments, Z¹ and Z² are different therapeutic agents targeting different biological pathways implicated in a common pathology. In some embodiments, Z¹ and Z² are different therapeutic agents, and the dendrimer conjugate of Formula (II) further comprises at least one targeting agent conjugated to the dendrimer. In some embodiments, Z¹ and Z² are different imaging agents. In some embodiments, Z¹ is a therapeutic agent, and Z² is a targeting agent. In some embodiments, Z¹ is an imaging agent, and Z² is a targeting agent. In some embodiments, at least one of Z¹ or Z² is a PPAR agonist (e.g., a PPAR-δ agonist).

Coupling Agents and Spacers

Dendrimer complexes can be formed of therapeutically active agents or compounds conjugated or bound to the dendrimers. Optionally, the active agents are conjugated to the dendrimers via one or more spacers/linkers via different linkages such as disulfide, ester, carbonate, carbamate, thioester, hydrazine, hydrazides, and amide linkages. The one or more spacers/linkers between a dendrimer and an agent can be designed to provide a releasable or non-releasable form of the dendrimer-active complexes in vivo. In some embodiments, the attachment occurs via an appropriate spacer that provides an ester bond between the agent and the dendrimer. In some embodiments, the attachment occurs via an appropriate spacer that provides an amide bond between the agent and the dendrimer. In some embodiments, one or more spacers/linkers between a dendrimer and an agent are added to achieve desired and effective release kinetics in vivo.

The spacer can be either a single chemical entity or two or more chemical entities linked together to bridge the polymer and the therapeutic agent or imaging agent. The spacers can include any small chemical entity, peptide or polymers having sulfhydryl, thiopyridine, succinimidyl, maleimide, vinylsulfone, and carbonate terminations.

The spacer can be chosen from among a class of compounds terminating in sulfhydryl, thiopyridine, succinimidyl, maleimide, vinylsulfone and carbonate group. The spacer can include thiopyridine terminated compounds such as dithiodipyridine, N-Succinimidyl 3-(2-pyridyldithio)-propionate (SPDP), Succinimidyl 6-(3-[2-pyridyldithio]-propionamido)hexanoate LC-SPDP or Sulfo-LC-SPDP. The spacer can also include peptides wherein the peptides are linear or cyclic essentially having sulfhydryl groups such as glutathione, homocysteine, cysteine and its derivatives, arg-gly-asp-cys (RGDC), cyclo(Arg-Gly-Asp-d-Phe-Cys) (c(RGDfC)), cyclo(Arg-Gly-Asp-D-Tyr-Cys), cyclo(Arg-Ala-Asp-d-Tyr-Cys). The spacer can be a mercapto acid derivative such as 3 mercapto propionic acid, mercapto acetic acid, 4 mercapto butyric acid, thiolan-2-one, 6 mercaptohexanoic acid, 5 mercapto valeric acid and other mercapto derivatives such as 2 mercaptoethanol and 2 mercaptoethylamine. The spacer can be thiosalicylic acid and its derivatives, (4-succinimidyloxycarbonyl-methyl-alpha-2-pyridylthio)toluene, (3-[2-pyridithio]propionyl hydrazide. The spacer can have maleimide terminations wherein the spacer includes polymer or small chemical entity such as bis-maleimido diethylene glycol and bis-maleimido triethylene glycol, Bis-Maleimidoethane, bismaleimidohexane. The spacer can include vinylsulfone such as 1,6-Hexane-bis-vinylsulfone. The spacer can include thioglycosides such as thioglucose. The spacer can be reduced proteins such as bovine serum albumin and human serum albumin, any thiol terminated compound capable of forming disulfide bonds. The spacer can include polyethylene glycol having maleimide, succinimidyl and thiol terminations.

Agents and/or targeting moiety can be either covalently attached or intra-molecularly dispersed or encapsulated in dendrimer. In some embodiments, the dendrimer is a PAMAM dendrimer up to generation 10, having carboxylic, hydroxyl, or amine terminations. In some embodiments, the dendrimer is linked to agents via a spacer ending in disulfide, ester or amide bonds.

Therapeutic, Prophylactic and Diagnostic Agents

In some embodiments, agents to be included in the particles (e.g., agents conjugated to dendrimers) to be delivered can be proteins or peptides, sugars or carbohydrates, nucleic acids or oligonucleotides, lipids, small molecules (e.g., molecular weight less than 2500 Daltons, less than 2000 Daltons, less than 1500 Daltons). The nucleic acid can be an oligonucleotide encoding a protein, for example, a DNA expression cassette or an mRNA. Representative oligonucleotides include siRNAs, microRNAs, DNA, and RNA. In some embodiments, the active agent is a therapeutic antibody.

In some embodiments, an agent is a nucleic acid, a nucleic acid analog, a small molecule having a molecular weight less than 2 kDa, less than 1 kDa, a peptidomimetic, a protein or peptide, carbohydrate or sugar, lipid, or surfactant, or a combination thereof. In some embodiments, agents include pharmaceutically acceptable, pharmacologically active derivatives of active agents, including, but not limited to, salts, esters, amides, prodrugs, active metabolites, and analogs.

Dendrimers have the advantage that multiple therapeutic, prophylactic, and/or diagnostic agents can be delivered with the same dendrimers. One or more types of active agents can be encapsulated, complexed or conjugated to the dendrimer. In one embodiment, the dendrimers are complexed with or conjugated to two or more different classes of agents, providing simultaneous delivery with different or independent release kinetics at the target site. In another embodiment, the dendrimers are covalently linked to at least one detectable moiety and at least one class of agents. In a further embodiment, dendrimer complexes each carrying different classes of agents are administered simultaneously for a combination treatment. Exemplary active agents include therapeutic agents useful for treating and preventing AKI and/or CKD.

In some embodiments, an agent is a therapeutic agent. In some embodiments, an agent is a diagnostic agent (e.g., an imaging agent). In some embodiments, a therapeutic agent is an agonist or an antagonist.

In some embodiments, an agent is an antagonist (e.g., an inhibitor). In some embodiments, a therapeutic agent is an inhibitor. In some embodiments, dendrimer compositions including one or more agents may inhibit or reduce the activity and/or quantity of pro-inflammatory (M1-like) macrophages, and/or pro-inflammatory cytokines in a diseased kidney by about 10%, 20%, 30%, 40%, 50%, 75%, 85%, 90%, 95%, or 99% from the activity and/or quantity of the same cells in the kidney of subjects that did not receive, or were not treated with the dendrimer compositions. In some embodiments, the inhibition and reduction are compared at mRNAs, proteins, cells, tissues and organs levels.

In some embodiments, an agent is an agonist. In some embodiments, an agonist refers to an agent that binds to, stimulates, increases, activates, facilitates, enhances activation, sensitizes or up regulates an activity or expression in vitro, ex vivo, or in vivo. For example, in some embodiments, an agent is a PPAR agonist (e.g., a PPAR-δ agonist). In some embodiments, a PPAR agonist is a molecule that binds a PPAR receptor with a half maximal effective concentration (EC₅₀) of less than 10 μM (e.g., less than 5 μM, less than 1 μM, less than 0.1 μM). In some embodiments, a PPAR agonist is a molecule that binds a PPAR receptor with an EC₅₀ of between about 0.1 nM and about 100 nM (e.g., 1-100 nM, 1-50 nM, 1-20 nM, 1-10 nM, 0.1-5 nM). Binding affinity can be evaluated to determine a value for EC₅₀, e.g., by an in vitro binding assay (e.g., fluorescence polarization, isothermal titration calorimetry, absorbance spectroscopy, and other methodologies known in the art).

Accordingly, in some embodiments, the disclosure provides a dendrimer conjugated to a PPAR agonist (e.g., a PPAR-δ agonist), and compositions and methods of use thereof.

A. Peroxisome Proliferator-Activated Receptor Delta (PPARδ) Agonists

Peroxisome-proliferator-activated receptors (PPARs) are nuclear hormone receptors including PPAR-α, PPAR-δ and PPAR-γ, which play an important role in regulating cancer cell proliferation, survival, apoptosis, and tumor growth. Peroxisome proliferator-activated receptor-delta (PPAR-δ), one of three members of the PPAR group in the nuclear receptor superfamily, is a ligand-activated transcription factor. PPAR-δ regulates important cellular metabolic functions that contribute to maintaining energy balance. PPAR-δ is especially important in regulating fatty acid uptake, transport, and β-oxidation as well as insulin secretion and sensitivity. These salutary PPAR-δ functions in normal cells are thought to protect against metabolic-syndrome-related diseases, such as obesity, dyslipidemia, insulin resistance/type 2 diabetes, hepatosteatosis, and atherosclerosis, and a multitude of physiological processes associated with glucose and lipid metabolism, inflammation and proliferation. It has been observed to be upregulated in several cancers. Although PPAR-δ is ubiquitously expressed, its expression level in different tissues varies depending on cell type and disease status.

As reported by Liu, et al. Int. J. Mol. Sci. 19(11):3339 (2018), many studies have revealed that PPARs are involved in regulation of inflammation. In some contexts, PPAR-δ has been reported to have anti-inflammatory functions. For example, the selective PPAR-δ agonist GW0742 alleviated inflammation in experimental autoimmune encephalomyelitis (EAE), while knockout of PPAR-δ aggravated EAE severity. PPAR-δ's antidiabetic functions also appear to be associated with reduced inflammatory signaling. In a rat model of type 2 diabetes, GW0742 was shown to reduce the proinflammatory cytokines tumor necrosis factor-α (TNF-α) and monocyte chemoattractant protein-1 (MCP-1) in liver tissues, in conjunction with reduced hepatic fat accumulation. GW0742 was also shown to inhibit streptozotocin-induced diabetic nephropathy in mice through a reduction of inflammatory mediators, including MCP-1 and osteopontin. A study using both the db/db (homozygous for the spontaneous db mutation in the leptin receptor gene (Lepr)) and high-fat-diet-induced obese diabetic mouse models showed that PPAR-δ is a key mediator in exercise-induced reduction of vascular inflammation.

PPAR-δ signaling appears to promote inflammation in other contexts. For example, PPAR-δ expression is increased in patients with psoriasis, a common immune-mediated disease primarily affecting the skin. In a transgenic mouse model, induction of PPAR-δ activation in the epidermis led to development of a psoriasis-like skin condition, which was correlated with increased IL-1 signaling and phosphorylation of STAT3. PPAR-δ signaling may also promote inflammation in some forms of arthritis. Mesenchymal stem cells (MSCs) have immunomodulatory properties that can limit inflammation. In a collagen-induced mouse model of arthritis, mice receiving MSCs with reduced PPAR-δ activity (MSCs harvested from PPAR-δ knockout mice or WT PPAR-δ MSCs pretreated with the PPAR-δ antagonist GSK3787) had better suppression of inflammatory immune responses, leading to improvements in arthritis scores. In the same study, inhibition of PPAR-δ with GSK3787 in human MSCs enhanced their ability to limit proliferation of peripheral blood mononuclear cells in co-culture experiments.

PPAR-δ agonists have been previously described. In some embodiments, the PPAR-δ agonists are indanylacetic acid derivatives carrying 4-thiazolyl-phenoxy tail groups as described in Rudolph J et al., J. Med. Chem. 2007, 50, 5, 984-1000 (2007). In some embodiments, dendrimers are complexed, covalently conjugated, or intra-molecularly dispersed or encapsulated with one or more PPAR-δ agonists selected from the following structures:

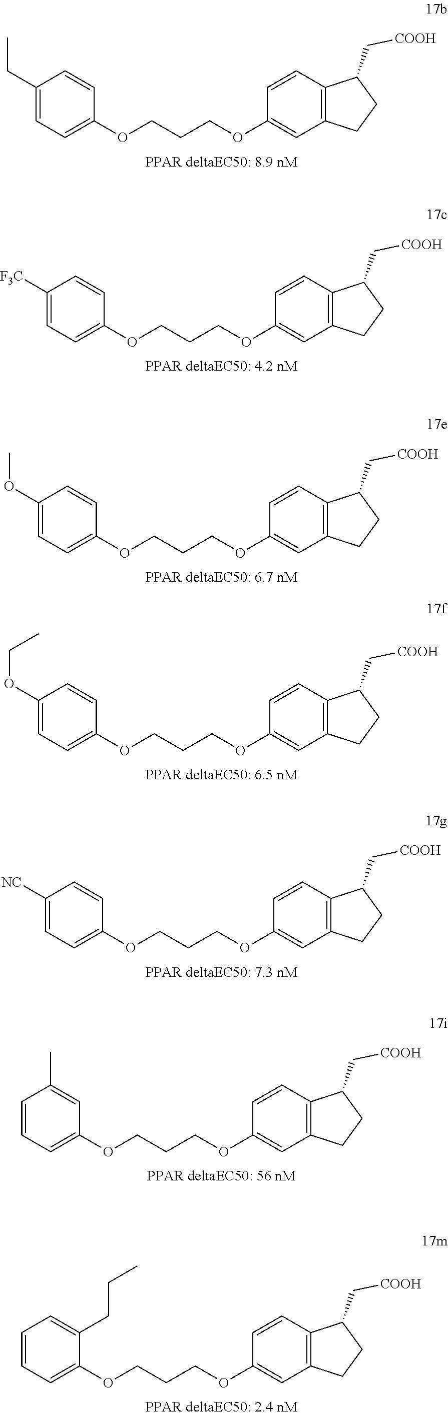

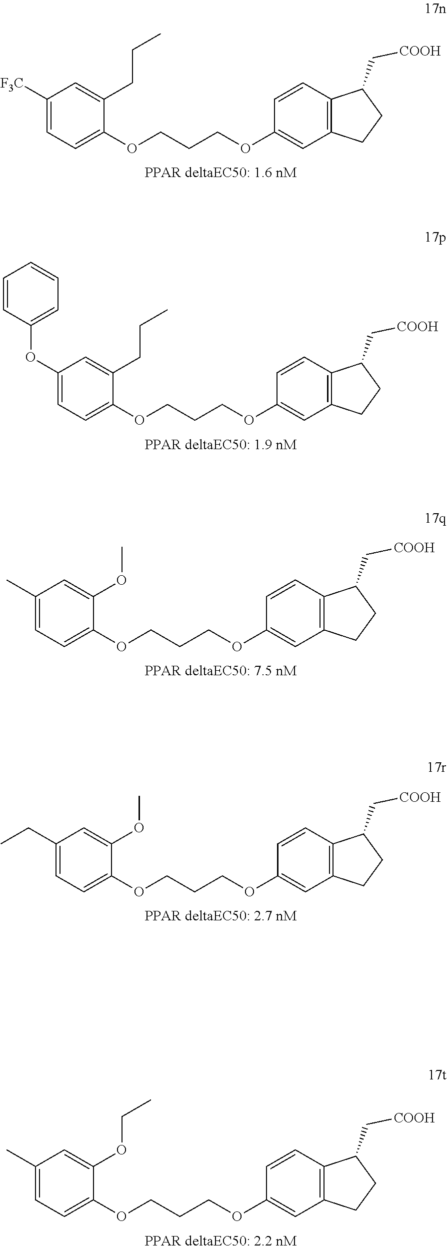 [SMILES: CCCc1cc(C(F)(F)F)ccc1OCCCOc1ccc2c(c1)CC[C@H]2CC(=O)O ; CCCc1cc(Oc2ccccc2)ccc1OCCCOc1ccc2c(c1)CC[C@H]2CC(=O)O ; COc1cc(C)ccc1OCCCOc1ccc2c(c1)CC[C@H]2CC(=O)O ; CCc1ccc(OCCCOc2ccc3c(c2)CC[C@H]3CC(=O)O)c(OC)c1 ; CCOc1cc(C)ccc1OCCCOc1ccc2c(c1)CC[C@H]2CC(=O)O]

In some embodiments, PPAR-δ agonists are functionalized, for example with ether, ester, or amide linkage, optionally, with one or more spacers/linkers, for ease of conjugation with the dendrimers and/or for desired release kinetics. In some embodiments, PPAR-δ agonists are functionalized to be non-cleavable or minimally cleavable from the dendrimers in vivo, for example via an ether bond, optionally with one or more spacers/linkers. Examples of conjugation of functionalized groups and/or linking moieties to PPAR-δ agonists are shown below.

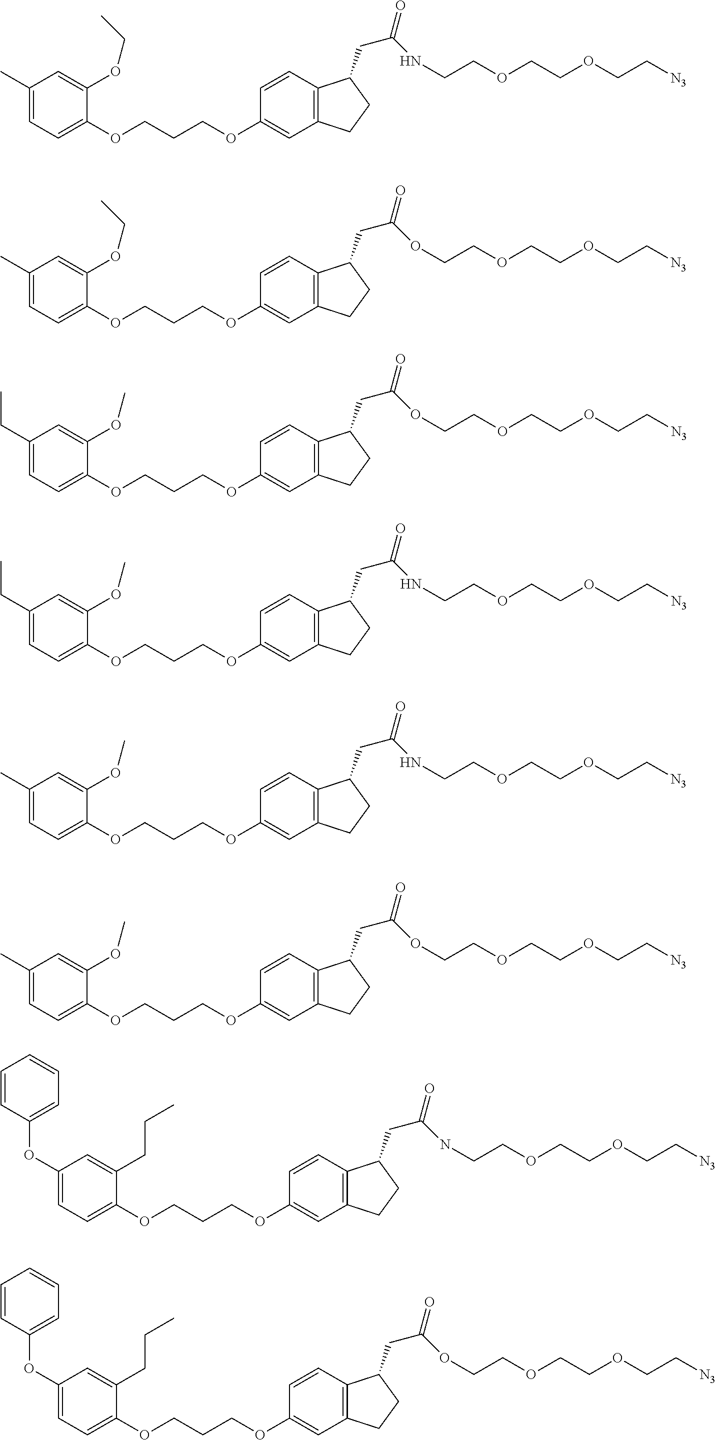

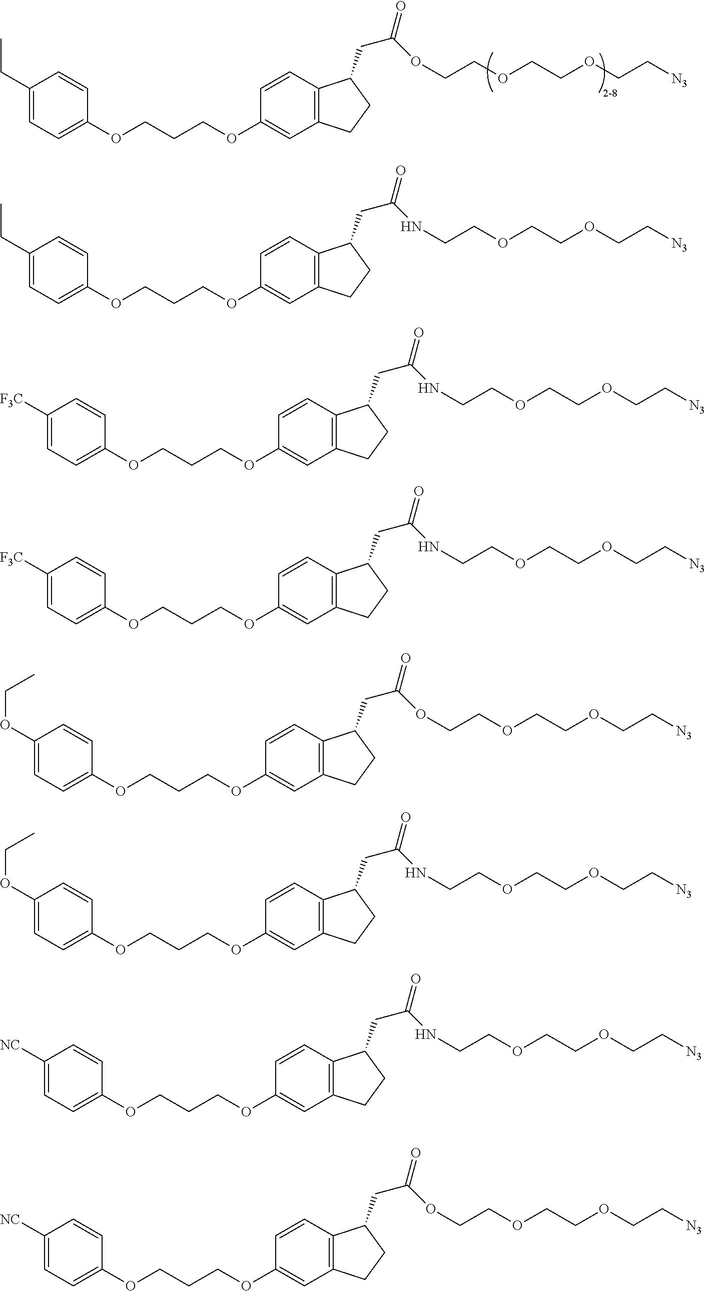

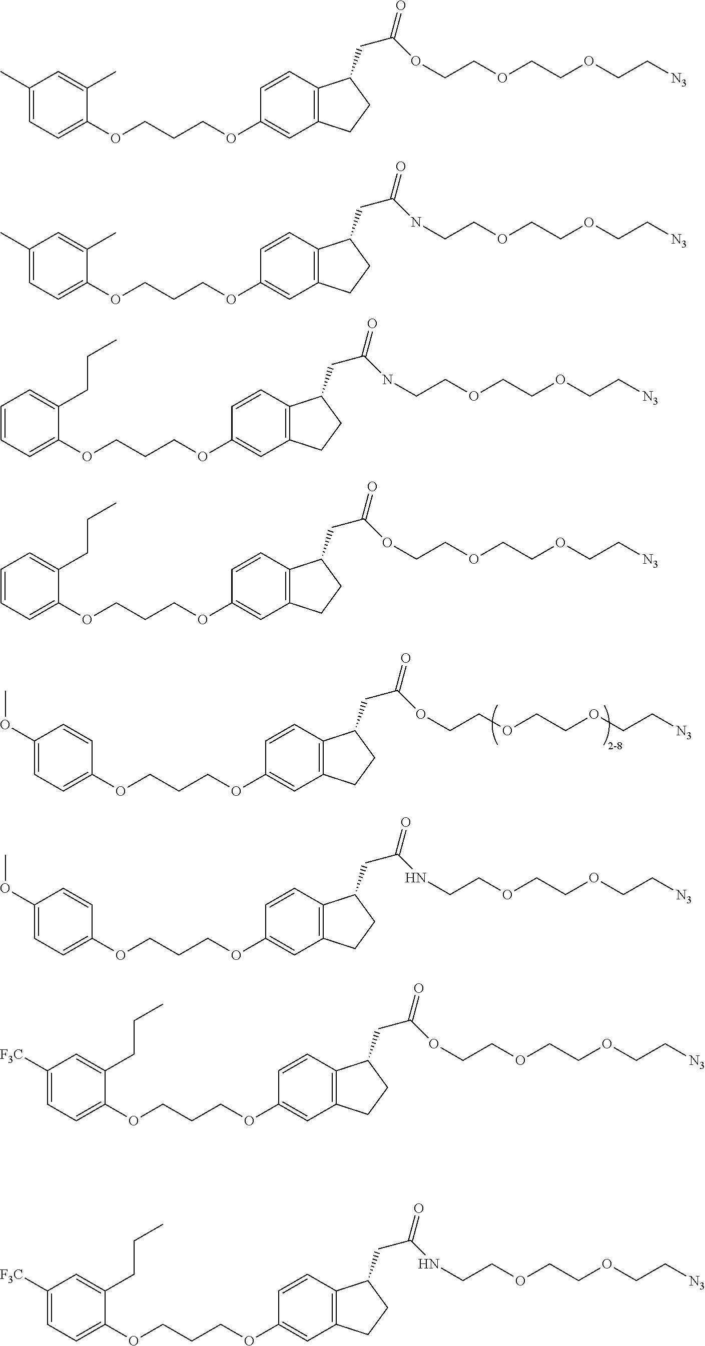

Additional examples of conjugation of functionalized groups and/or linking moieties to PPAR-δ agonists are shown below. See also, Rudolph J et al., J. Med. Chem. 2007, 50, 5, 984-1000 (2007).

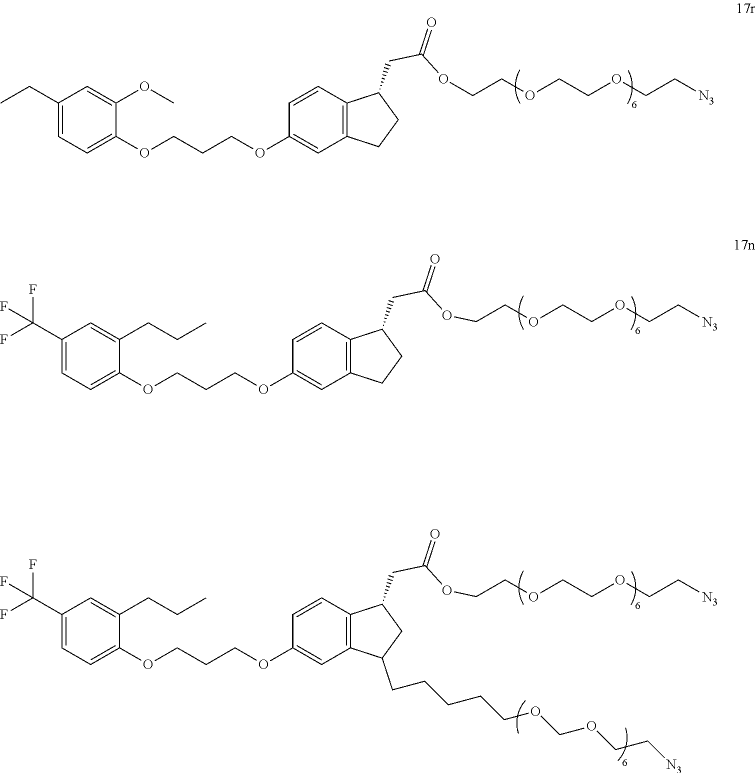

In some embodiments, dendrimers are complexed, covalently conjugated, or intra-molecularly dispersed or encapsulated with one or more PPAR-δ agonists selected from the following structures:

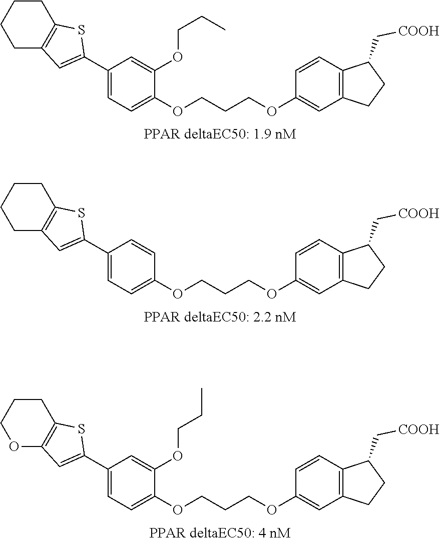 [SMILES: CCCOc1cc(-c2cc3c(s2)CCCC3)ccc1OCCCOc1ccc2c(c1)CC[C@H]2CC(=O)O ; O=C(O)C[C@@H]1CCc2cc(OCCCOc3ccc(-c4cc5c(s4)CCCC5)cc3)ccc21 ; CCCOc1cc(-c2cc3c(s2)CCCO3)ccc1OCCCOc1ccc2c(c1)CC[C@H]2CC(=O)O]

Further examples of conjugation of functionalized groups and/or linking moieties to PPAR-δ agonists are shown below.

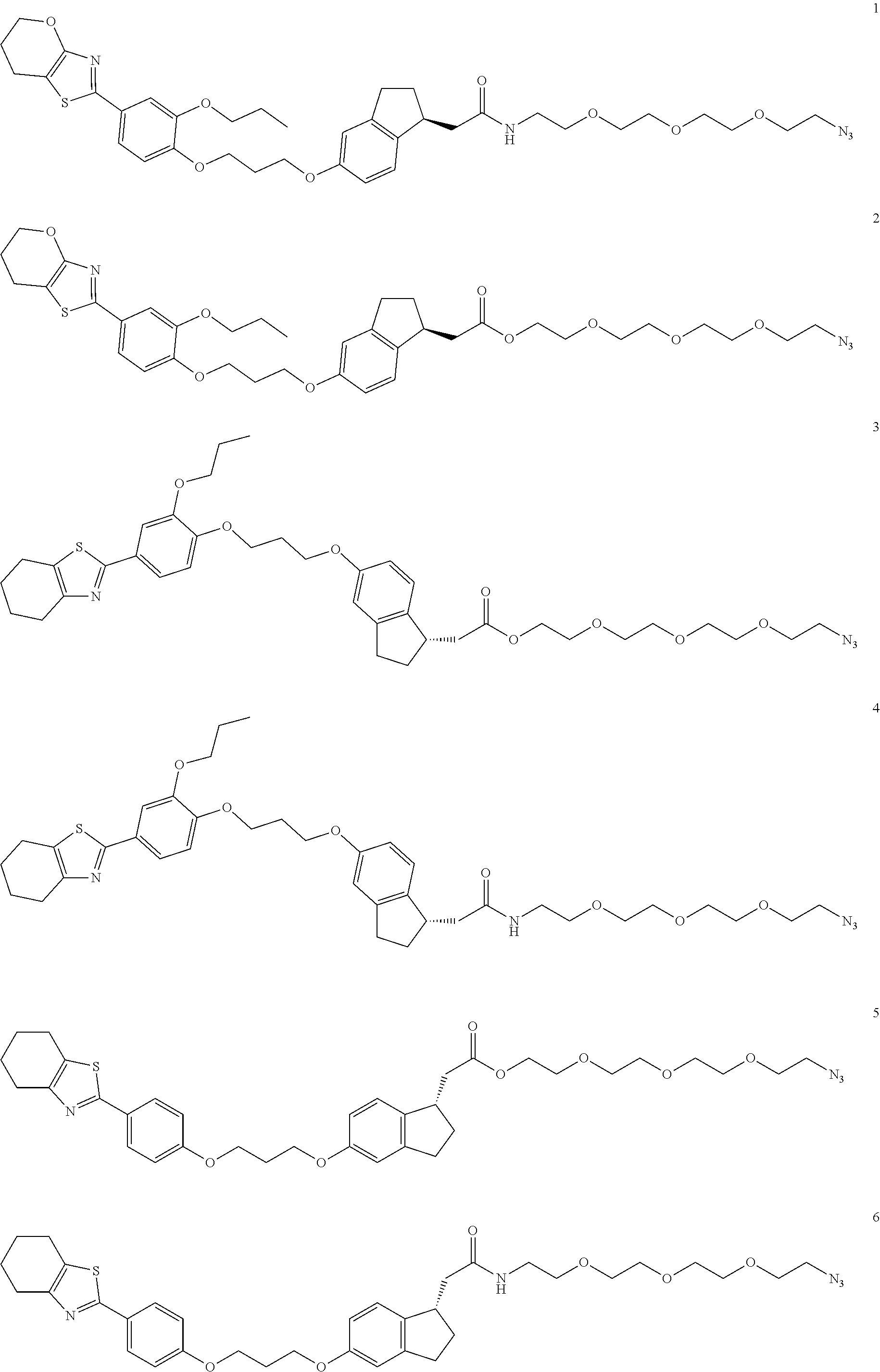

Additional examples of PPAR-δ agonists have been previously described, for example, by Ham J et al., Eur J Med Chem. 53:190-202 (2012). In some embodiments, dendrimers are complexed, covalently conjugated, or intra-molecularly dispersed or encapsulated with one or more PPAR-δ agonists selected from the following structures:

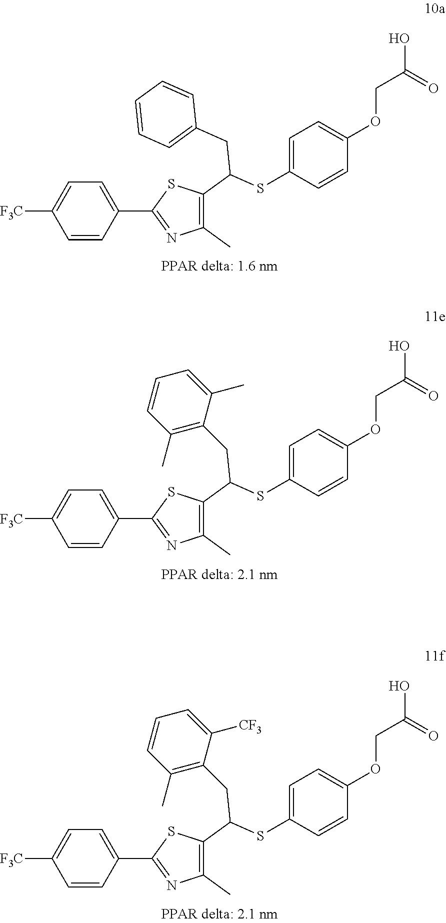 [SMILES: Cc1nc(-c2ccc(C(F)(F)F)cc2)sc1C(Cc1ccccc1)Sc1ccc(OCC(=O)O)cc1 ; Cc1cccc(C)c1CC(Sc1ccc(OCC(=O)O)cc1)c1sc(-c2ccc(C(F)(F)F)cc2)nc1C ; Cc1cccc(C(F)(F)F)c1CC(Sc1ccc(OCC(=O)O)cc1)c1sc(-c2ccc(C(F)(F)F)cc2)nc1C]

Further examples of conjugation of functionalized groups and/or linking moieties to PPAR-δ agonists suitable for conjugation to dendrimers are shown below:

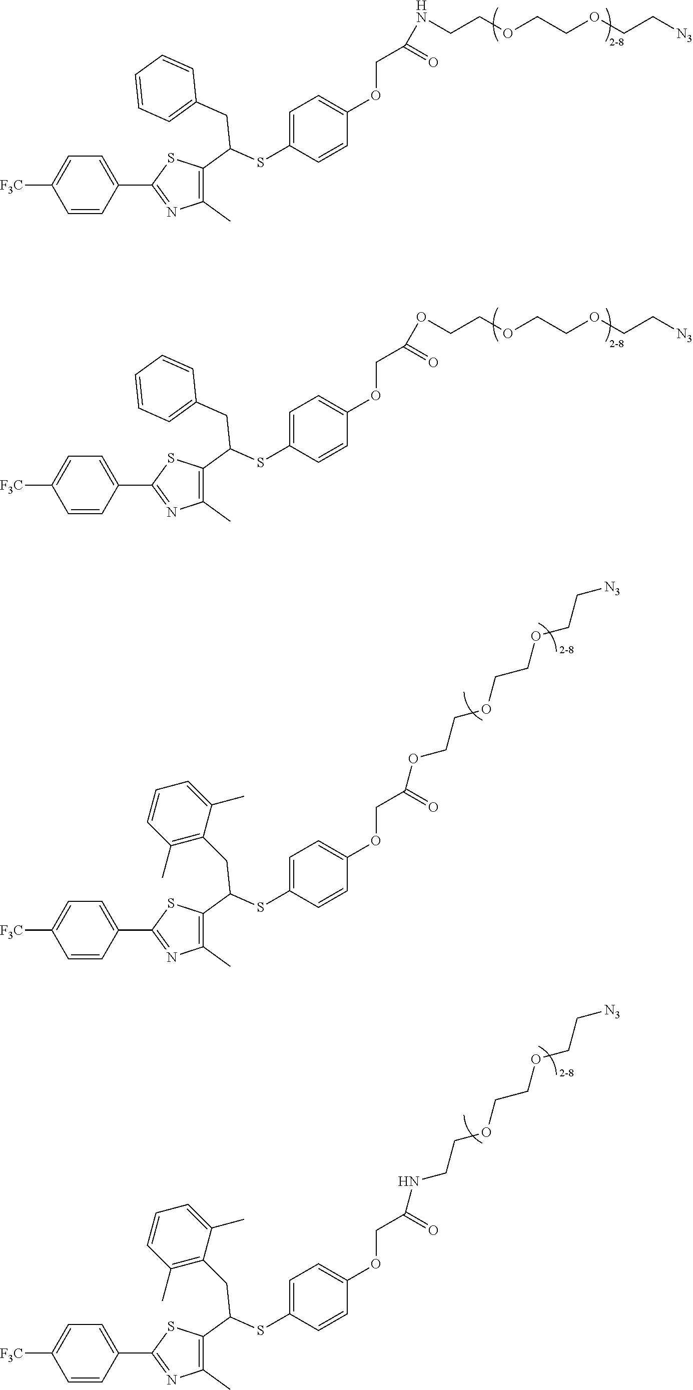

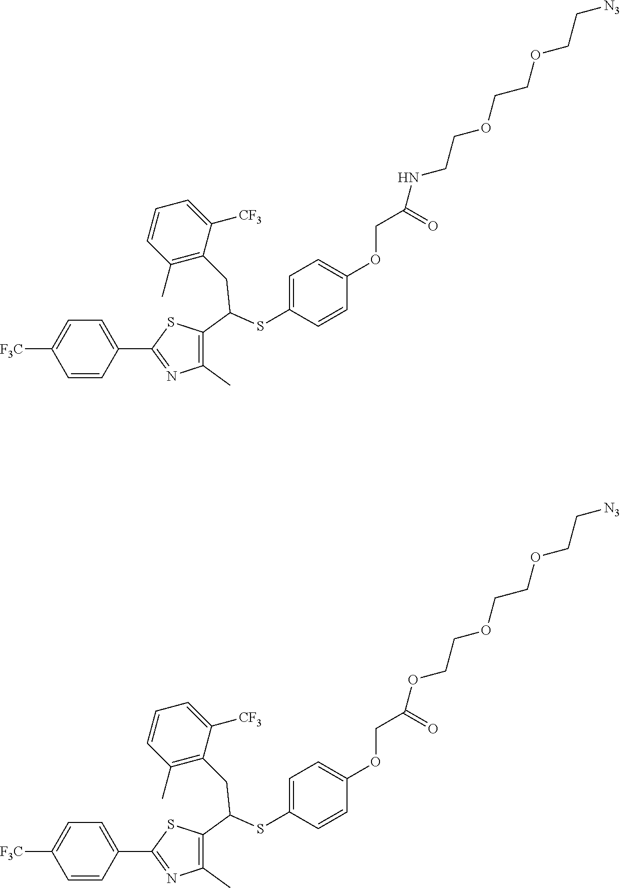 [SMILES: Cc1cccc(C(F)(F)F)c1CC(Sc1ccc(OCC(=O)NCCOCCOCCN=[N+]=[N-])cc1)c1sc(-c2ccc(C(F)(F)F)cc2)nc1C ; Cc1cccc(C(F)(F)F)c1CC(Sc1ccc(OCC(=O)OCCOCCOCCN=[N+]=[N-])cc1)c1sc(-c2ccc(C(F)(F)F)cc2)nc1C]

In one embodiment, dendrimers are complexed, covalently conjugated, or intra-molecularly dispersed or encapsulated with PPAR-δ agonist GW0742. Examples of conjugation of a GW0742 as GW0742-amide derivative and GW0742-ester derivative are shown below:

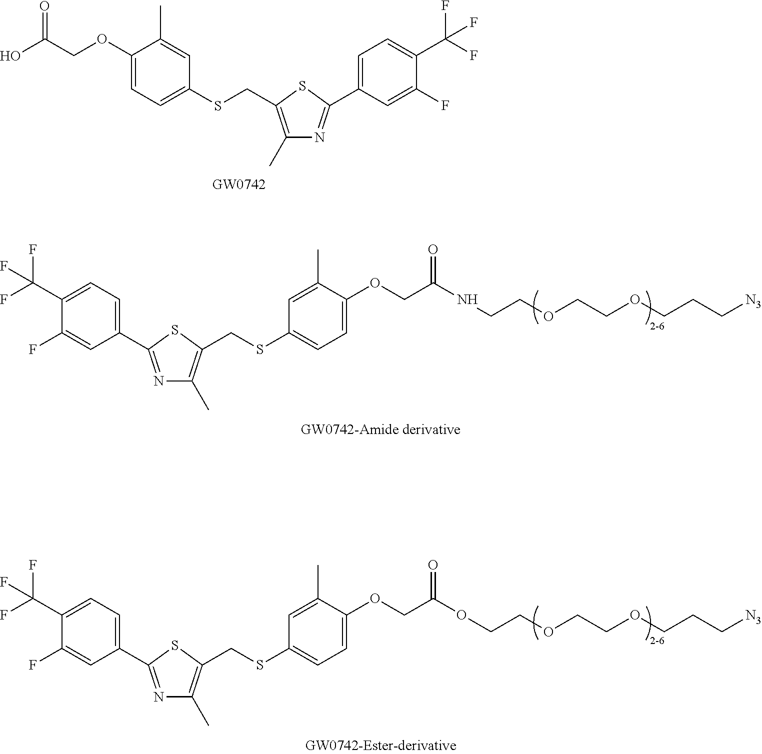

In another embodiment, dendrimers are complexed, covalently conjugated, or intra-molecularly dispersed or encapsulated with PPAR-δ agonist GW501516. Examples of conjugation of a GW0742 as GW0742-amide derivative and GW0742-ester derivative are shown below:

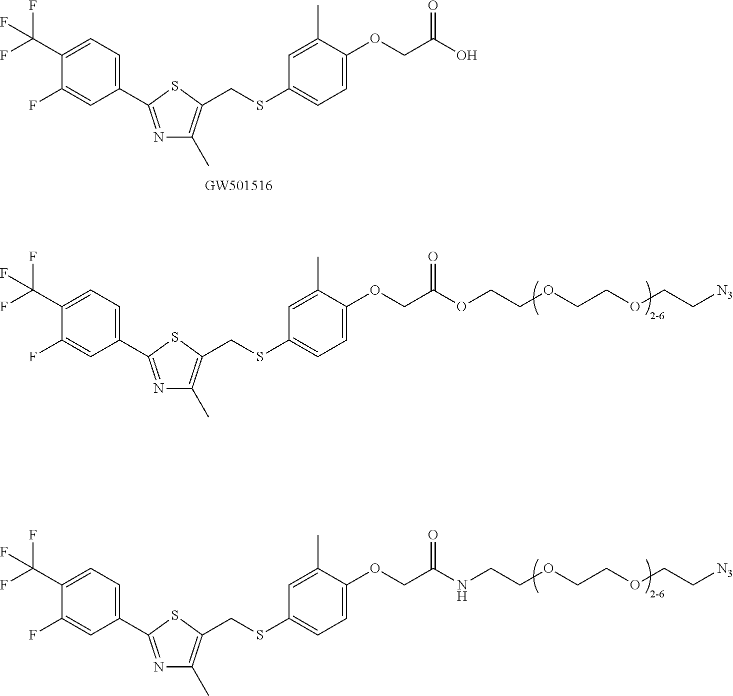

B. Anti-Inflammatory Agents

In some embodiments, the compositions include one or more anti-inflammatory agents. Anti-inflammatory agents reduce inflammation and include steroidal and non-steroidal drugs.

In some embodiments, an anti-inflammatory agent is an antioxidant drug, such as N-acetylcysteine.

Examples of steroidal anti-inflammatory drugs include, without limitation, Triamicinolone acetonide, dexamethasone, methylprednisolone, hydrocortisone acetate, cortisone, diflucortolone, difluprednate, Flucinonide, alclometasone, difluprednate, triamcinolone diacetate, betamethasone, betamethasone valerate, beclometasone and their salts and prodrugs. Glucocortiocoid steroidal antiinflammatories include prednisone, dexamethasone, and corticosteroids such as fluocinolone acetonide and methylprednisolone.

Examples of non steroidal drugs can be classified into NSAIDS and COX-2 inhibitors. These include ibufenac, acetylsalicylic acid, benoxaprofen, naproxen, alminoproxen, bucloxic acid, ibuprofen, celecoxib, carprofen, etodolac, flufenamic acid, flubiprofen, indomethacin, isoxepac, ketoprofen, mefanemic acid, oxaprozen, oxpinac, parecoxib, phenylbutazone, piroxicam, sulindac, suprofen, tiaprofenic acid, tolmetin, tramadol, valdecoxib salts and their prodrugs.

Gold and conjugates thereof can be used as anti-inflammatory agents.

In some embodiments, an anti-inflammatory agent is an immune-modulating drug. Examples of immune-modulating drugs include cyclosporine, tacrolimus and rapamycin. In some embodiments, anti-inflammatory agents are biologic drugs that block the action of one or more immune cell types such as T cells, or block proteins in the immune system, such as tumor necrosis factor-alpha (TNF-alpha), interleukin 17-A, interleukin-12, and interleukin-23.

In some embodiments, an anti-inflammatory agent is an SGLT2 inhibitor, an LPA1 receptor antagonist or LPA1 signaling pathway inhibitor, a vasopressin V2-receptor antagonist, an endothelin receptor antagonist, or a uric acid transporter inhibitor. Examples of SGLT2 inhibitors include: Phlorizin, T-1095, canagliflozin, dapagliflozin, ipragliflozin, tofogliflozin, empagliflozin, luseogliflozin, ertugliflozin, and remogliflozin etabonate. Examples of LPA1 receptor antagonists or LPA1 signaling pathway inhibitors include: BMS-986202, BMS-986020, VPC12249, AM966, AM095, Ki16425, and Ki16198. Examples of vasopressin V2-receptor antagonists include Lixivaptan, Tolvaptan, Satavaptan, and Mozavaptan. Examples of endothelin receptor antagonists include Sitaxentan, Ambrisentan, Macitentan, and Zibotentan. Examples of uric acid transporter inhibitors include probenecid, sulfinpyrazone, benzbromarone, lesinurad, RDEA3170, SHR4640, URC-102 and FYU-981.

In some embodiments, the anti-inflammatory drug is a synthetic or natural anti-inflammatory protein. Antibodies specific to select immune components can be added to immunosuppressive therapy. In some embodiments, the anti-inflammatory drug is an anti-T cell antibody (e.g., anti-thymocyte globulin or Anti-lymphocyte globulin), anti-IL-2Rα receptor antibody (e.g., basiliximab or daclizumab), or anti-CD20 antibody (e.g., rituximab).

Many inflammatory diseases may be linked to pathologically elevated signaling via the receptor for lipopolysaccharide (LPS), toll-like receptor 4 (TLR4). Thus, in some embodiments, the active agents are one or more TLR4 inhibitors.

In some embodiments, the one or more anti-inflammatory drugs are released from the dendrimer complexes after administration to a mammalian subject in an amount effective to inhibit inflammation for at least 1 day, 2 days, 3 days, 4 days, 5 days, 6 days, preferably at least a week, 2 weeks, or 3 weeks, more preferably at least a month, two months, three months, four months, five months, or six months.

C. Agents for Treatment of Kidney Disease, Hypertension and Other Disorders

In some embodiments, the dendrimers are used to deliver one or more additional active agents, particularly one or more therapeutic, prophylactic and/or diagnostic agents to prevent or treat one or more symptoms of kidney injuries and/or associated diseases or conditions such as infections, sepsis, ischemia-reperfusion injury, diabetic complications, hypertension, obesity, and/or autoimmunity diabetes, high blood pressure, heart failure, kidney diseases, liver diseases, and cancers.

In some embodiments, other agents can be incorporated such as chemotherapeutic agents, anti-angiogenic agents, and anti-excitotoxic agents, such as valproic acid, D-aminophosphonovalerate, D-aminophosphonoheptanoate, inhibitors of glutamate formation/release such as baclofen, NMDA receptor antagonists, ranibizumab, and anti-VEGF agents including aflibercept, and immunomodulators such as rapamycin.

Other therapeutic agents that may be delivered include uric acid transporter (URAT1) inhibitors (e.g. verinurad), vasopression V2-receptor antagonists (e.g. tolvaptan), endothelin receptor antagonists (e.g. atrasentan), subtype 2 sodium-glucose transport protein (SGLT2) inhibitor (e.g. canagliflozin), and LPA1 receptor antagonists.

In some embodiments, the active agent is an anti-infectious agent. Exemplary anti-infectious agents include antiviral agents, antibacterial agents, antiparasitic agents, and anti-fungal agents.

In some embodiments, the dendrimers deliver one or more therapeutic agents that have been shown to have efficacy for treating and preventing AKI and/or CKD.

D. Diagnostic Agents

In some cases, the agent may include a diagnostic agent. Examples of diagnostic agents include paramagnetic molecules, fluorescent compounds, magnetic molecules, and radionuclides, x-ray imaging agents, and contrast media. Examples of other suitable contrast agents include gases or gas emitting compounds, which are radiopaque. Dendrimer complexes can further include agents useful for determining the location of administered compositions. Agents useful for this purpose include fluorescent tags, radionuclides and contrast agents.

Exemplary diagnostic agents include dyes, fluorescent dyes, near infra-red dyes, SPECT imaging agents, PET imaging agents and radioisotopes.

In further embodiments, a singular dendrimer complex composition can simultaneously treat and/or diagnose a disease or a condition at one or more locations in the body.

In some aspects, the disclosure provides a dendrimer conjugate comprising a dendrimer having at least one imaging agent at one or more terminal positions of the dendrimer. In some embodiments, a dendrimer conjugate comprising an imaging agent can be used for diagnostic, therapeutic, or labeling purposes. In some embodiments, an imaging agent is a paramagnetic molecule, a fluorescent compound, a magnetic molecule, a radionuclide, an x-ray imaging agents, or a contrast agent. In some embodiments, a contrast agent is a gas or gas-emitting compound, which is radioopaque. In some embodiments, a dendrimer conjugate comprising an imaging agent can be used for determining the location of administered compositions. Imaging agents useful for this purpose include, without limitation, fluorescent tags, radionuclides, and contrast agents. Examples of imaging agents useful for diagnostic purposes include, without limitation, dyes, fluorescent dyes, near infrared dyes, SPECT imaging agents, PET imaging agents, and radioisotopes. Examples of dyes include, without limitation, carbocyanine, indocarbocyanine, oxacarbocyanine, thüicarbocyanine and merocyanine, polymethine, coumarin, rhodamine, xanthene, fluorescein, boron-dipyrromethane (BODIPY), Cy5, Cy5.5, Cy7, VivoTag-680, VivoTag-S680, VivoTag-S750, AlexaFluor660, AlexaFluor680, AlexaFluor700, AlexaFluor750, AlexaFluor790, Dy677, Dy676, Dy682, Dy752, Dy780, DyLight547, Dylight647, HiLyte Fluor 647, HiLyte Fluor 680, HiLyte Fluor 750, IRDye 800CW, IRDye 800RS, IRDye 700DX, ADS780WS, ADS830WS, and ADS832WS.

In some embodiments, a dendrimer conjugate comprises a radionuclide reporter appropriate for imaging by scintigraphy, single-photon emission computed tomography (SPECT), or positron emission tomography (PET). In some embodiments, a dendrimer conjugate comprises a radionuclide appropriate for radiotherapy. In some embodiments, a dendrimer conjugate comprises a contrast agent for imaging by magnetic resonance imaging (MRI). In some embodiments, a dendrimer conjugate comprises a chelator for a radionuclide or an MRI contrast agent useful for diagnostic imaging, and a chelator useful for radiotherapy. Accordingly, in some embodiments, a single dendrimer/imaging agent conjugate can simultaneously treat and diagnose a disease or a condition at one or more locations in the body. In some embodiments, a dendrimer conjugate comprises a radioactively labeled SPECT, or scintigraphic imaging agents that have a suitable amount of radioactivity.

Suitable imaging agents can be selected based on a particular imaging methodology. For example, in some embodiments, an imaging agent is a near infrared fluorescent dye for optical imaging, a Gadolinium chelate for MRI imaging, a radionuclide for PET or SPECT imaging, or a gold nanoparticle for CT imaging.

In some embodiments, a dendrimer conjugate comprises one or more imaging agents for PET imaging, such as one or more radionuclides. PET is a technique that uses a special camera and a computer to detect small amounts of radioactive radiotracers or radiopharmaceuticals in vivo, to evaluate organ and tissue functions (e.g., to detect early onset of a disease).

PET involves the detection of gamma rays in the form of annihilation photons from short-lived positron emitting radioactive isotopes including, but not limited to, ¹⁸F with a half-life of approximately 110 minutes, ¹¹C with a half-life of approximately twenty minutes, ¹³N with a half-life of approximately ten minutes, and ¹⁵O with a half-life of approximately two minutes, using coincidence detection. Accordingly, in some embodiments, examples of imaging agents for use in PET imaging include, without limitation, one or more of the various positron emitting metal ions, such as ⁵¹Mn, ⁵²Fe, ⁶⁰Cu, ⁶⁸Ga, ⁷²As, ⁹⁴mTc, or ¹¹⁰In. In some embodiments, an imaging agent is a radionuclide selected from ¹⁸F, ¹²⁴I, ¹²⁵I, ¹³¹I, ¹²³I, ⁷⁷Br, and ⁷⁶Br. Examples of metal radionuclides for scintigraphy or radiotherapy include, without limitation, ^{99m}Tc, ⁵¹Cr, ⁶⁷Gr, ⁶⁸Ga, ⁴⁷Sc, ⁵¹Cr, ¹⁶⁷Tm, ¹⁴¹Ce, ¹¹¹In, ¹⁶⁸Yb, ¹⁷⁵Yb, ⁹⁰Y, ⁸⁸Y, ¹⁵³Sm, ¹⁶⁶Ho, ¹⁶⁵Dy, ¹⁶⁶Dy, ⁶²Cu, ⁶⁴Cu, ⁶⁷Cu, ⁹⁷Ru, ¹⁰³Ru, ¹⁸⁶Re, ¹⁸⁸Re, ²⁰³Pb, ²¹¹Bi, ²¹²Bi, ²¹³Bi, ²¹⁴Bi, ¹⁰⁵Rh, ¹⁰⁹Pd, ¹¹⁷mSn, ¹⁴⁹Pm, ¹⁶¹Tb, ¹⁷⁷Lu, ²²⁵Ac, ¹⁹⁸Au and ¹⁹⁹Au. The choice of metal will be determined based on the desired therapeutic or diagnostic application. For example, for diagnostic purposes, in some embodiments, useful radionuclides include ⁶⁴Cu, ⁶⁷Ga, ⁶⁸Ga, ^{99m}Tc, and ¹¹¹In. For therapeutic purposes, in some embodiments, useful radionuclides include ⁶⁴Cu, ⁹⁰Y, ¹⁰⁵Rh, ¹¹¹In, ¹¹⁷mSn, ¹⁴⁹Pm, ¹⁵³Sm, ¹⁶¹Tb, ¹⁶⁶Tb, ¹⁶⁶Dy, ¹⁶⁶Ho, ¹⁷⁵Yb, ¹⁷⁷Lu, ²²⁵Ac, ^{186/188}Re, and ¹⁹⁹Au.

In some embodiments, an imaging agent is technetium-99m (^{99m}Tc). In some embodiments, ^{99m}Tc is useful for diagnostic applications because of its low cost, availability, imaging properties, and high specific activity. The nuclear and radioactive properties of ^{99m}Tc make this isotope useful for scintigraphic imaging. This isotope has a single photon energy of 140 keV and a radioactive half-life of about 6 hours, and is readily available from a ⁹⁹Mo-^{99m}Tc generator. In some embodiments, radionuclides useful for PET imaging include ¹⁸F, 4-[¹⁸F]fluorobenzaldehyde (¹⁸FB), Al[¹⁸F]-NOTA, ⁶⁸Ga-DOTA, and ⁶⁸Ga-NOTA. In some embodiments, ¹⁵³Sm can be used as an imaging agent with chelators such as ethylenediaminetetramethylenephosphonic acid (EDTMP) or 1,4,7,10-tetraazacyclododecanetetramethylenephosphonic acid (DOTMP).

MRI can be used to assess brain disease, spinal disorder, angiography, cardiac function, and musculoskeletal damage, among other uses. MRI does not require the use of ionizing radiation, and scans can be performed at any chosen orientation. MRI provides full three-dimensional capabilities, high soft-tissue contrast, high spatial resolution, and is adept at morphological and functional imaging. Accordingly, in some embodiments, a dendrimer comprises one or more imaging agents for MRI, such as one or more MRI contrast agents. Examples of MRI contrast agents are known in the art and include, without limitation, Gd, Mn, BaSO₄, iron oxides, and iron platinum.

### II. Pharmaceutical Formulations

Pharmaceutical compositions including dendrimers and one or more active agents such as peroxisome proliferator-activated receptor delta (PPAR-δ) agonists may be formulated in a conventional manner using one or more physiologically acceptable carriers including excipients and auxiliaries which facilitate processing of the active compounds into preparations which can be used pharmaceutically. Proper formulation is dependent upon the route of administration chosen. In preferred embodiments, the compositions are formulated for parenteral delivery. In some embodiments, the compositions are formulated for subcutaneous injection. Typically the compositions will be formulated in sterile saline or buffered solution for injection into the tissues or cells to be treated. The compositions can be stored lyophilized in single use vials for rehydration immediately before use. Other means for rehydration and administration are known to those skilled in the art.

Pharmaceutical formulations contain one or more dendrimer complexes in combination with one or more pharmaceutically acceptable excipients. Representative excipients include solvents, diluents, pH modifying agents, preservatives, antioxidants, suspending agents, wetting agents, viscosity modifiers, tonicity agents, stabilizing agents, and combinations thereof. Suitable pharmaceutically acceptable excipients are preferably selected from materials which are generally recognized as safe (GRAS), and may be administered to an individual without causing undesirable biological side effects or unwanted interactions. See, for example, Remington's Pharmaceutical Sciences, 20th ed., Lippincott Williams & Wilkins, Baltimore, M D, 2000, p. 704.

The compositions are preferably formulated in dosage unit form for ease of administration and uniformity of dosage. The phrase “dosage unit form” refers to a physically discrete unit of conjugate appropriate for the patient to be treated. It will be understood, however, that the total single administration of the compositions will be decided by the attending physician within the scope of sound medical judgment. The therapeutically effective dose can be estimated initially either in cell culture assays or in animal models, usually mice, rabbits, dogs, or pigs. The animal model is also used to achieve a desirable concentration range and route of administration. Such information should then be useful to determine useful doses and routes for administration in humans. Therapeutic efficacy and toxicity of conjugates can be determined by standard pharmaceutical procedures in cell cultures or experimental animals, e.g., ED50 (the dose is therapeutically effective in 50% of the population) and LD50 (the dose is lethal to 50% of the population). The dose ratio of toxic to therapeutic effects is the therapeutic index and it can be expressed as the ratio, LD50/ED50. Pharmaceutical compositions which exhibit large therapeutic indices are preferred. The data obtained from cell culture assays and animal studies can be used in formulating a range of dosages for human use.

Pharmaceutical compositions formulated for administration by parenteral (intramuscular, intraperitoneal, intravenous or subcutaneous injection) and enteral routes of administration are described.

The phrases “parenteral administration” and “administered parenterally” are art-recognized terms, and include modes of administration other than enteral and topical administration, such as injections, and include, without limitation, intravenous, intramuscular, intrapleural, intravascular, intrapericardial, intraarterial, intrathecal, intracapsular, intraorbital, intracardiac, intradennal, intraperitoneal, transtracheal, subcutaneous, subcuticular, intraarticular, subcapsular, subarachnoid, intraspinal and intrastemal injection and infusion. The dendrimers can be administered parenterally, for example, by subdural, intravenous, intrathecal, intraventricular, intraarterial, intra-amniotic, intraperitoneal, or subcutaneous routes. In preferred embodiments, the dendrimer compositions are administered via subcutaneous injection.

For liquid formulations, pharmaceutically acceptable carriers may be, for example, aqueous or non-aqueous solutions, suspensions, emulsions or oils. Parenteral vehicles (for subcutaneous, intravenous, intraarterial, or intramuscular injection) include, for example, sodium chloride solution, Ringer's dextrose, dextrose and sodium chloride, lactated Ringer's and fixed oils. Examples of non-aqueous solvents are propylene glycol, polyethylene glycol, and injectable organic esters such as ethyl oleate. Aqueous carriers include, for example, water, alcoholic/aqueous solutions, cyclodextrins, emulsions or suspensions, including saline and buffered media. The dendrimers can also be administered in an emulsion, for example, water in oil. Examples of oils are those of petroleum, animal, vegetable, or synthetic origin, petrolatum, and mineral. Suitable fatty acids for use in parenteral formulations include, for example, oleic acid, stearic acid, and isostearic acid. Ethyl oleate and isopropyl myristate are examples of suitable fatty acid esters.

Formulations suitable for parenteral administration can include antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. Intravenous vehicles can include fluid and nutrient replenishers, electrolyte replenishers such as those based on Ringer's dextrose. In general, water, saline, aqueous dextrose and related sugar solutions, and glycols such as propylene glycols or polyethylene glycol are preferred liquid carriers, particularly for injectable solutions.

Injectable pharmaceutical carriers for injectable compositions are well-known to those of ordinary skill in the art (see, e.g., Pharmaceutics and Pharmacy Practice, J. B. Lippincott Company, Philadelphia, PA, Banker and Chalmers, eds., pages 238-250 (1982), and ASHP Handbook on Injectable Drugs, Trissel, 15th ed., pages 622-630 (2009)).

The compositions can be administered enterally. The carriers or diluents may be solid carriers such as capsule or tablets or diluents for solid formulations, liquid carriers or diluents for liquid formulations, or mixtures thereof.

For liquid formulations, pharmaceutically acceptable carriers may be, for example, aqueous or non-aqueous solutions, suspensions, emulsions or oils. Examples of non-aqueous solvents are propylene glycol, polyethylene glycol, and injectable organic esters such as ethyl oleate. Aqueous carriers include, for example, water, alcoholic/aqueous solutions, cyclodextrins, emulsions or suspensions, including saline and buffered media.

Examples of oils are those of petroleum, animal, vegetable, or synthetic origin, for example, peanut oil, soybean oil, mineral oil, olive oil, sunflower oil, fish-liver oil, sesame oil, cottonseed oil, corn oil, olive, petrolatum, and mineral. Suitable fatty acids for use in parenteral formulations include, for example, oleic acid, stearic acid, and isostearic acid. Ethyl oleate and isopropyl myristate are examples of suitable fatty acid esters.

Vehicles include, for example, sodium chloride solution, Ringer's dextrose, dextrose and sodium chloride, lactated Ringer's and fixed oils. Formulations include, for example, aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. Vehicles can include, for example, fluid and nutrient replenishers, electrolyte replenishers such as those based on Ringer's dextrose. In general, water, saline, aqueous dextrose and related sugar solutions are preferred liquid carriers. These can also be formulated with proteins, fats, saccharides and other components of infant formulas.

In preferred embodiments, the compositions are formulated for oral administration. Oral formulations may be in the form of chewing gum, gel strips, tablets, capsules or lozenges. Encapsulating substances for the preparation of enteric-coated oral formulations include cellulose acetate phthalate, polyvinyl acetate phthalate, hydroxypropyl methylcellulose phthalate and methacrylic acid ester copolymers. Solid oral formulations such as capsules or tablets are preferred. Elixirs and syrups also are well known oral formulations.

### III. Methods of Use

Methods of using the dendrimer compositions for treating or preventing diseases or disorders in a subject are described. The dendrimer compositions can be used to treat, prevent, and/or diagnose one or more symptoms of one or more kidney injuries, disorders, and/or diseases in a subject in need thereof. Methods for treating or preventing one or more symptoms of one or more kidney injuries, disorders, and/or diseases include administering to the subject dendrimers complexed, covalently conjugated, or intra-molecularly dispersed or encapsulated with one or more therapeutic or prophylactic agents, in an amount effective to treat, alleviate or prevent one or more symptoms of one or more kidney injuries, disorders, and/or diseases. In some embodiments, the dendrimer compositions including one or more anti-inflammatory agents and/or PPAR-δ agonists, or formulations thereof are administered in an amount effective to treat or prevent one or more symptoms of one or more kidney injuries, disorders, and/or diseases, for example, reducing inflammation in the kidney.

In one embodiment, methods for treating or preventing one or more kidney injuries, disorders, and/or diseases include administering to the subject compositions including G4, G5, or G6 PAMAM dendrimers covalently conjugated to one or more PPAR-δ agonists, in an amount effective to treat or prevent one or more symptoms of one or more kidney injuries, disorders, and/or diseases.

In some embodiments, the dendrimer complexes are used to treat AKI, for example, those caused by impaired blood flow to the kidneys, caused by direct damage to the kidneys, or caused by urine blockage in the kidneys. The methods typically include administering to a subject in a need thereof an effective amount of a composition including dendrimer and one or more agents to treat and/or alleviate one or more symptoms associated with the kidney disorders and/or diseases.

Methods for treating or ameliorating one or more symptoms of kidney injuries and/or diseases are described. In particular, the compositions are used in an amount effective for treating or ameliorating one or more symptoms of acute kidney injury (AKI) and chronic kidney disease (CKD), for example, those associated with a condition that slows blood flow to the kidneys. Diseases and conditions that may slow blood flow to the kidneys and lead to kidney injury include blood or fluid loss, blood pressure medications, heart attack, heart disease, infection, liver failure, use of non-steroidal antiinflammatory drugs or related drugs, severe allergic reaction (anaphylaxis), severe burns, severe dehydration. Acute kidney failure usually occurs in connection with another medical condition or event. Conditions that can increase the risk of acute kidney failure include being hospitalized (especially for a serious condition that requires intensive care), advanced age, blockages in the blood vessels in the arms or legs (peripheral artery disease), diabetes, high blood pressure, heart failure, kidney diseases, liver diseases, certain cancers and their treatments. Thus, in some embodiments, the dendrimer compositions are administered in an amount effective to reduce mortality rate, to reduce occurrence of organ failure, to reduce hospitalization time.

Methods for reducing tubular damage, tubular epithelial flattening, tubular dilatation, and tubular epithelial cell necrosis, and particularly of proximal tubules in the kidneys are also described. In some embodiments, the dendrimer compositions reduce and/or inhibit tubular damage, tubular epithelial flattening, tubular dilatation, and tubular epithelial cell necrosis and/or apoptosis in the diseased kidneys. In some embodiments, the dendrimer compositions promote recovery of tubular cell integrity and function in the diseased kidney.

Inflammation and immune system activation represent a common underlying characteristic for both AKI and CKD. Cellular damage and its associated molecular products are thought to be key triggers for inflammation after acute tissue injury (Chen G Y et al., Nat Rev Immunol 10: 826-837 (2010)). Within the kidney, renal tubular epithelial cells are extremely susceptible to intrinsic oxidative stress, particularly during the reperfusion phase of ischemia/reperfusion (IR) (Medzhitov R, Cell 140: 771-776 (2010); Kurts C, et al., Nat Rev Immunol 13: 738-753 (2013)). Necrotic cells release damage-associated molecular patterns, such as high-mobility group box 1, histones, heat shock proteins, fibronectin, and biglycan into the extracellular spaces, which subsequently, activate pattern recognition receptors, such as toll-like receptors (TLRs), and nucleotide-binding oligomerization domain-like receptors, such as the nucleotide-binding oligomerization domain-, LRR-, and pyrin domain-containing 3 inflammasome, expressed in epithelial and endothelial cells, dendritic cells (DCs), monocytes/macrophages, and lymphocytes (Anders H J et al, J Am Soc Nephrol 25: 1387-1400 (2014); Vallés P G, et al., Int J Nephrol Renovasc Dis 7: 241-251, 2014). Activated renal parenchyma cells and DCs also secrete chemokines, including CXCL1, CXCL8, CCL2, and CCL5, that promote acute neutrophil- and monocyte/macrophage-dependent inflammatory responses in AKI (Bolisetty S, et al., Kidney Int 75: 674-676 (2009)). Time-dependent changes in the expression of pro-inflammatory (e.g., TNF-α, IFN-γ, IL-6, IL-1β, IL-23, IL-17, C3, C5a, and C5b) and anti-inflammatory (e.g., IL-4, TGF-β, IL-10, heme oxygenase 1, resolvins, and protectin D1) mediators by resident and recruited cell populations are important determinants of the injury and repair phases. Under ideal conditions, a fine balance between inflammatory and anti-inflammatory factors ensures robust tissue repair and a return of homeostatic conditions. However, AKI often results in an abnormal repair process as a result of prolonged hypoxia and sustained secretion of profibrotic cytokine (e.g., IL-13 and TGF-β1), leading to post-AK fibrosis and chronic renal dysfunction (Anders H J, et al., J Am Soc Nephrol 25: 1387-1400, (2014)).

In some embodiments, the dendrimer compositions are used in an amount effective to decrease production of pro-inflammatory cytokines, and/or promote generation of anti-inflammatory cytokines, and/or anti-inflammatory phenotype of one or more immune cell types. In other embodiments, the compositions are used to suppress pro-inflammatory and promote anti-inflammatory properties of one or more immune cells involved in the one or more kidney injuries, conditions, and/or diseases to be treated.

In some embodiments, the compositions are administered in an amount effective to inhibit or reduce one or more pro-inflammatory cytokines such as TNF-α, IFN-γ, IL-6, IL-1β, IL-23, IL-17; to inhibit or reduce one or more chemokines and/or chemokines receptors such as CCR2 and CX3CR1; and/or to inhibit or reduce reactive oxygen species and nitric oxide in the disease/damaged kidney. In further embodiments, the compositions can increase production of anti-inflammatory cytokines such as IL-4, TGF-β, IL-10.

Pro-inflammatory cells or inflammatory cells refer to immune cells that promote pro-inflammatory activities, secretion of pro-inflammatory cytokines such as IL-12, IFN-γ, and TNF-α, or a combination thereof. Exemplary pro-inflammatory cells including pro-inflammatory M1 macrophages or classically activated macrophages (CAMs). In some embodiments, methods for depleting, inhibiting or reducing pro-inflammatory macrophages or classically activated macrophages (M1-like macrophages) in a subject, for example, by blocking proliferation, migration, or activation of the pro-inflammatory macrophages in the diseased kidney, are described. In some embodiments, the methods administer to a subject dendrimer complexes including one or more active agents an effective amount to deplete, inhibit, or reduce the number or activities of the pro-inflammatory M1 macrophages by 10%, 20%, 30%, 40%, 50%, 60%, 70%, 80%, 90%, 100%, 150%, 200%, 250%, 300%, or more than 300% relative to such levels before treatment with the dendrimer compositions.

In some embodiments, the compositions and formulations thereof are used to reduce/inhibit an inflammatory response in a subject in need thereof by administering an effective amount of the compositions to reduce activation, proliferation and/or recruitment of one or more pro-inflammatory cells, and/or enhance activation, proliferation and/or recruitment of one or more suppressive immune cells are provided. In some embodiments, the pro-inflammatory cells are pro-inflammatory M1 macrophages. In further embodiments, the suppressive immune cells are M2-like macrophages. Thus, in some embodiments, the compositions can promote the switch from a pro-inflammatory phenotype (M1 macrophage) to an anti-inflammatory state (M2 macrophage) at one or more diseased tissues/organs including the kidney, by reducing proliferation and/or generation of M1 macrophage, to enhance activation, proliferation and/or generation of M2 macrophages, and/or to increase the ratio of M2 macrophages to M1 macrophages, effective to ameliorate one or more symptoms of an inflammatory condition in the kidney All the methods described can include the step of identifying and selecting a subject in need of treatment, or a subject who would benefit from administration with the compositions.

A. Methods for Treating Renal Ischemia Reperfusion Injury

Ischemia/reperfusion injury (IRI) is caused by a sudden temporary impairment of the blood flow to the particular organ. IRI usually is associated with a robust inflammatory and oxidative stress response to hypoxia and reperfusion which disturbs the organ function. Renal IR induced acute kidney injury (AKI) contributes to high morbidity and mortality rate in a wide range of injuries. In ischemic kidney and subsequent of re-oxygenation, generation of reactive oxygen species (ROS) at reperfusion phase initiates a cascade of deleterious cellular responses leading to inflammation, cell death, and acute kidney failure.

General, the compositions and methods of treatment thereof are suitable for treating one or more kidney injuries, conditions, and/or diseases that are directly or indirectly result of renal IR, in particular renal IR induced acute kidney injury (AKI) and chronic kidney disease (CKD).

In preferred embodiments, the dendrimers are used to treat or prevent AKI and CKD, in particular renal IR induced AKI and CKD.

Acute kidney injury (AKI) is one of a number of conditions that affect kidney structure and function. AKI is defined by an abrupt decrease in kidney function that includes, but is not limited to, ARF. It is a broad clinical syndrome encompassing various etiologies, including specific kidney diseases (e.g., acute interstitial nephritis, acute glomerular and vasculitic renal diseases); non-specific conditions (e.g, ischemia, toxic injury); as well as extrarenal pathology (e.g., prerenal azotemia, and acute postrenal obstructive nephropathy). More than one of these conditions may coexist in the same patient. Furthermore, because the manifestations and clinical consequences of AKI can be quite similar (even indistinguishable) regardless of whether the etiology is predominantly within the kidney or predominantly from outside stresses on the kidney, the syndrome of AKI encompasses both direct injury to the kidney as well as acute impairment of function. AKI clinically manifests as a reversible acute increase in nitrogen waste products, measured by blood urea nitrogen (BUN) and serum creatinine levels, over the course of hours to weeks. The spectrum of injury ranges from mild to advanced, sometimes requiring renal replacement therapy.

Other biomarkers as diagnostic indicators of kidney injury include glycosuria; increased proteinuria; or increased urinary N-acetyl-β-d-glucosamininidase (NAG), γ-GT, or AP levels, urinary kidney injury molecule-1 (KIM-1), and urinary human neutrophil gelatinase-associated lipocalin (NGAL).

Accordingly, the composition is administered in an amount effective to reduce or alleviate glycosuria, increased proteinuria, to reduce serum levels of creatinine and/or blood urea nitrogen (BUN), to reduce urinary N-acetyl-β-d-glucosamininidase (NAG), γ-GT, and/or AP levels, and/or to reduce urine NGAL and/or KIM-1 content.

Glomerular filtration rate (GFR) is the best measure of kidney function. The GFR is the number used to determine the stage of kidney disease. A mathematical formula using the person's age, race, gender and their serum creatinine is used to calculate GFR. A doctor will order a blood test to measure the serum creatinine level. Creatinine is a waste product that comes from muscle activity. When kidneys are working well they remove creatinine from the blood. As kidney function slows, blood levels of creatinine rise.

The different stages of CKD form a continuum. The stages of CKD are classified as follows:
Stage 1: Kidney damage with normal or increased GFR (>90 mL/min/1.73 m²)
Stage 2: Mild reduction in GFR (60-89 mL/min/1.73 m²)
Stage 3a: Moderate reduction in GFR (45-59 mL/min/1.73 m²)
Stage 3b: Moderate reduction in GFR (30-44 mL/min/1.73 m²)
Stage 4: Severe reduction in GFR (15-29 mL/min/1.73 m²)
Stage 5: Kidney failure (GFR<15 mL/min/1.73 m² or dialysis)

Accordingly, the dendrimer compositions or formulations thereof are administered to a mammalian subject, preferably human, in an amount effective to reduce tubular damage in the kidney, to reduce blood urea nitrogen (BUN) and/or creatinine (CR), and/or improve or increase in glomerular filtration rate (GFR).

Dosage and dosing regimens are dependent on the severity and location of the disorder or injury and/or methods of administration, and can be determined by those skilled in the art. A therapeutically effective amount of the dendrimer composition used in the treatment of kidney injuries and/or diseases is typically sufficient to reduce or alleviate one or more symptoms of kidney injuries and/or diseases.

Preferably the active agents do not target or otherwise modulate the activity or quantity of healthy cells not within or associated with the diseased/damaged tissue, or do so at a reduced level compared to cells associated with the diseased/damaged kidney. In this way, by-products and other side effects associated with the compositions are reduced.

A pharmaceutical composition including a therapeutically effective amount of the dendrimer compositions and a pharmaceutically acceptable diluent, carrier or excipient is described. In some embodiments, the pharmaceutical compositions includes an effective amount of hydroxyl-terminated PAMAM dendrimers conjugated to N-acetyl cysteine. In some embodiments, dosage ranges suitable for use are between about 0.1 mg/kg and about 100 mg/kg, inclusive; between about 0.5 mg/kg and about 40 mg/kg, inclusive; between about 1.0 mg/kg and about 20 mg/kg, inclusive; and between about 2.0 mg/kg and about 10 mg/kg, inclusive.

Dosage forms of the pharmaceutical composition including the dendrimer compositions are also provided. “Dosage form” refers to the physical form of a dose of a therapeutic compound, such as a capsule or vial, intended to be administered to a patient. The term “dosage unit” refers to the amount of the therapeutic compounds to be administered to a patient in a single dose. In some embodiments, the dosage unit suitable for use are (assuming the weight of an average adult patient is 70 kg) between 5 mg/dosage unit and about 7000 mg/dosage unit, inclusive; between about 35 mg/dosage unit and about 2800 mg/dosage unit, inclusive; and between about 70 mg/dosage unit and about 1400 mg/dosage unit, inclusive; and between about 140 mg/dosage unit and about 700 mg/dosage unit, inclusive.

The actual effective amounts of dendrimer complex can vary according to factors including the specific active agent administered, the particular composition formulated, the mode of administration, and the age, weight, condition of the subject being treated, as well as the route of administration and the disease or disorder. The subjects are preferably humans. Generally, the dosage may be lower for intravenous injection or infusion.

In general, the timing and frequency of administration will be adjusted to balance the efficacy of a given treatment or diagnostic schedule with the side-effects of the given delivery system. Exemplary dosing frequencies include continuous infusion, single and multiple administrations such as hourly, daily, weekly, monthly or yearly dosing.

In some embodiments, dosages are administered once, twice, or three times daily, or every other day, two days, three days, four days, five days, or six days to a human. In some embodiments, dosages are administered about once or twice every week, every two weeks, every three weeks, or every four weeks. In some embodiments, dosages are administered about once or twice every month, every two months, every three months, every four months, every five months, or every six months.

It will be understood by those of ordinary skill that a dosing regimen can be any length of time sufficient to treat the disorder in the subject. In some embodiments, the regimen includes one or more cycles of a round of therapy followed by a drug holiday (e.g., no drug). The drug holiday can be 1, 2, 3, 4, 5, 6, or 7 days; or 1, 2, 3, 4 weeks, or 1, 2, 3, 4, 5, or 6 months.

The effect of the dendrimer compositions including one or more agents can be compared to a control. Suitable controls are known in the art and include, for example, an untreated subject, or a placebo-treated subject. A typical control is a comparison of a condition or symptom of a subject prior to and after administration of the targeted agent. The condition or symptom can be a biochemical, molecular, physiological, or pathological readout. For example, the effect of the composition on a particular symptom, pharmacologic, or physiologic indicator can be compared to an untreated subject, or the condition of the subject prior to treatment. In some embodiments, the symptom, pharmacologic, or physiologic indicator is measured in a subject prior to treatment, and again one or more times after treatment is initiated. In some embodiments, the control is a reference level, or average determined based on measuring the symptom, pharmacologic, or physiologic indicator in one or more subjects that do not have the disease or condition to be treated (e.g., healthy subjects). In some embodiments, the effect of the treatment is compared to a conventional treatment that is known the art.

B. Combination Therapies and Procedures

The compositions can be administered alone or in combination with one or more conventional therapies. In some embodiments, the conventional therapy includes administration of one or more of the compositions in combination with one or more additional active agents. The combination therapies can include administration of the active agents together in the same admixture, or in separate admixtures. Therefore, in some embodiments, the pharmaceutical composition includes two, three, or more active agents. Such formulations typically include an effective amount of an agent targeting the site of treatment. The additional active agent(s) can have the same or different mechanisms of action. In some embodiments, the combination results in an additive effect on the treatment of the renal condition. In some embodiments, the combinations result in a more than additive effect on the treatment of the disease or disorder.

The additional therapy or procedure can be simultaneous or sequential with the administration of the dendrimer composition. In some embodiments, the additional therapy is performed between drug cycles or during a drug holiday that is part of the compositions dosage regime.

Exemplary additional therapies or procedures include intravenous (IV) fluids in case of a lack of fluids in the blood, medications (diuretics) to cause body to expel extra fluids if too much fluid causes swelling in the limbs, medications to control blood potassium such as calcium, glucose or sodium polystyrene sulfonate (KIONEX®), medications to restore blood calcium levels such as an infusion of calcium, and/or hemodialysis to remove toxin in the body.

In some embodiments, the compositions and methods are used prior to or in conjunction, subsequent to, or in alternation with treatment with one or more additional therapies or procedures.

### IV. Kits

The compositions can be packaged in kit. The kit can include a single dose or a plurality of doses of a composition including one or more active agents encapsulated in, associated with, or conjugated to a dendrimer, and instructions for administering the compositions. Specifically, the instructions direct that an effective amount of the composition be administered to an individual with a particular renal condition/disease such AKI or CKD as indicated. The composition can be formulated as described above with reference to a particular treatment method and can be packaged in any convenient manner.

The present disclosure will be further understood by reference to the following non-limiting examples.

### EXAMPLES

### Example 1: Renal Ischemia/Reperfusion Injury Model in Experimental Diabetic Rats

Materials and Methods

Diabetes was induced in Wistar rats by administration of streptozotocin (STZ) (70 mg/kg) as a single intraperitoneal (IP) injection. Four days after STZ injection, the blood glucose levels were measured. Rats with a blood glucose of >16.7 mM were allocated to four groups (G1-G4; n=3/group). After six weeks, ischemia reperfusion injury (IRI) was inflicted with 60 min ischemia(I)/6 hr reperfusion (R) (G2), or 45 min I/24 hr R (G3 & G4). A sham surgery was performed as a control (G1). Hydroxyl dendrimer labeled with Cy5 (D-Cy5) was administered via intraperitoneal injection 1 hr after IRI in G1, G2 and G3 and 12 hr after IRI in G4. Renal function was assessed by clinical chemistry, glomerular filtration rate (GFR), and kidney injury biomarkers. Rats were euthanized 6 hr (G2) or 24 hr (G1, G3, G4) after surgery. Kidneys were fixed in 10% formalin, embedded in paraffin, sectioned, and evaluated for tubular damage and tubular epithelial cell necrosis. Sections were also stained by DAPI and anti-CD68 antibody (macrophage).

STZ-Induced Type I Diabetic Rat Modeling
1) A single dose of STZ (70 mg/kg) was injected intraperitoneally into Wistar rats (SPF) to induce type I diabetes.
2) 4 days after STZ injection, peripheral blood samples were collected for blood glucose level determination. Diabetes is defined by a blood glucose level of >16.7 mmol/L.

Bilateral Renal Ischemia/Reperfusion Model Establishment
1) IRI modeling was performed 6 weeks post-STZ injection.
2) Before IRI surgery, rats were anesthetized by using inhalation anesthesia with isoflurane (2-5% in air).
3) The bilateral abdominal wall was opened in layers to expose the renal artery.
4) Renal ischemia was induced via occlusion of bilateral renal arteries using a non-traumatic artery clamp, followed by reperfusion for the assigned length of time for each group. Two types of surgery conditions were used: 60 min I/6 hr R and 45 min I/24 hr R. Sham-operated rats underwent surgical procedures identical to those used for I/R except that artery clamps were not applied.
5) All animals were maintained under a temperature-controlled pad (37° C.) until awareness and then were transferred to home cages.

Grouping and Treatment

Based on body weights, all rats were assigned to four groups in the BioBook system (IDBS). A unique number was assigned to each animal. Prior to the allocation of animals to treatment groups, cages were labeled with cards identifying study number, species/strain, sex, cage number, and animal number. After allocation to treatment groups, the cages were labeled with cards which are color-coded and identified treatment groups as well as the information outlined above. Group allocation was documented in the randomization records. Cages were stratified within the racks to reduce the effect of any environmental influences on the study. Grouping according to the scheme below.

TABLE 1
Experiment groups and dosing regimen

| | | | | | | Dose & | |
|---|---|---|---|---|---|---|---|
| | | | D- | | Dosing | Dosing | |
| Group | No. | STZ | Cy5 | R.O.A | Time | Vol. | Euthanization |
| 1 | 3 | Yes | Yes | i.p. | 1 hr after | 55 mg/kg; | 24 hr after |
| | | | | | surgery | 10 mL/kg | surgery |
| 2 | 3 | Yes | Yes | i.p. | 1 hr after | 55 mg/kg; | 6 hr after |
| | | | | | the start of | 10 mL/kg | surgery |
| | | | | | reperfusion | | |
| 3 | 3 | Yes | Yes | i.p. | 1 hr after | 55 mg/kg; | 24 hr after |
| | | | | | the start of | 10 mL/kg | surgery |
| | | | | | reperfusion | | |
| 4 | 3 | Yes | Yes | i.p. | 12 hr after | 55 mg/kg; | 24 hr after |
| | | | | | the start of | 10 mL/kg | surgery |
| | | | | | reperfusion | | |

Clinical Observations & Body Weight

Animals were closely monitored for body weight changes (every other day), health status, and possible death. All operated rats were observed by the experimenters and datasheets were used to record any animal abnormality. Animals would be euthanized if the animal's body weight decreased markedly (by more than 25% in 48 hrs) considering animal welfare. The euthanized body would be dissected in time and an anatomical report would be provided. No rats died during this study.

Animal Euthanasia and Sample Collection

Urine collection: After D-Cy5 injection, animals were placed into conventional metabolic cages and urine samples were collected until euthanasia.

Peripheral blood collection: All rats were euthanized by i.p. injection of 100 mg/kg sodium pentobarbital lidocaine. Peripheral blood was collected, and serum was prepared (Blood was placed at room temperature for 30 min and centrifuged at 4° C., 5000 rpm for 5 minutes). The serum was then stored at −80° C. for later biochemistry analysis.

Kidney collection: After euthanasia, animals were perfused with saline through the left ventricle. Bilateral kidneys were collected, and gross kidney images were taken. Each kidney was separated into two parts along the hilum. Two parts of the kidney (one from the left kidney and the other from the right kidney) were processed into frozen sections for IF imaging. And, the other two parts from bilateral kidneys were fixed with 10% formalin and processed into paraffin blocks for histopathological analysis.

Blood Glucose Measurement

Blood glucose measurement was performed four days post-STZ injection and on the day before surgery with a glucometer.

Biochemistry Analysis

Renal function was assessed by measuring serum creatinine, urine creatinine, and BUN levels using Hitachi 7060 Biochemical Analyser. GFR was measured using the comparative values of creatinine in blood and urine. Urine NGAL and KIM-1 levels were measured by ELISA kits following the instruction provided with the kit.

Kidney Pathology Analysis

Kidney tissues that were fixed in 10% formalin were used for pathological staining. After embedded with paraffin, kidney tissue sections of 4 μm thickness were de-paraffinized, then stained with H&E or PAS for light microscopy evaluation according to CBL standard procedures. One section from each animal was used for H&E and PAS staining, respectively. The pathological changes were examined by in-house pathologists to determine the extent of renal injury. The sections were scored over 5 randomly selected fields (1 mm²) according to the following scoring system:

TABLE 2
Criteria for evaluating tubular damage (degree of tubular
epithelial flattening and tubular dilatation)

| | Percentage of tubular epithelial |
|---|---|
| Grade | flattening and tubular dilatation |
| 0 | 0% |
| 1 | 0-10% |
| 2 | 10-25% |
| 3 | 25-50% |
| 4 | 50-75% |
| 5 | >75% |

TABLE 3
Criteria for evaluating tubular epithelial cell necrosis

| | Percentage of tubular epithelial cell |
|---|---|
| Grade | necrosis |
| 0 | Denotes no change |
| 1 | <25% (mild) |
| 2 | 25-50% (moderate) |
| 3 | >50% (severe) |

TABLE 4
Criteria for evaluating the renal interstitial inflammation

| Grade | Percentage of tubular involvement |
|---|---|
| 0 | None |
| 1 | Mild |
| 2 | Moderate |
| 3 | Severe |

Co-Localization Study

Kidneys were placed in a sucrose gradient and were then sectioned into 10 μm slices. One section of bilateral kidneys from each animal was evaluated. Kidneys were stained with DAPI to visualize cell nuclei and ED1 to visualize macrophages. Sections were incubated with primary anti-ED1 antibody (Seritec.INC), followed by secondary antibody Alexa Fluor 488 Phalloidin (CST). Sections were counterstained with DAPI, coverslipped with DAKO fluorescence mounting medium, and stored at −20° C. Images were captured over 5 randomly selected fields at 400× magnification. The number of cells co-stained with ED1 and D-Cy5 was counted and the positive area of D-Cy5 per unit area in the kidney was calculated.

Statistical Analysis

Results are expressed as the mean±SEM and statistically evaluated using One-Way ANOVA (Dunnett's multiple comparison test) on GraphPad prism 7.0. Differences between groups were considered significant with P value<0.05.

### Results

The study used a renal IRI model, the STZ-induced type 1 diabetic model in Wistar rats. Based on the model development results, the modeling surgery condition and dosing regimen can be determined for in vivo efficacy evaluation of anti-inflammatory drug compounds.

Model Establishment and Compound Administration

Type 1 diabetes was induced by a single i.p. injection of STZ (70 mg/kg). Four days after STZ injection, the blood glucose levels were measured, and animals with a blood glucose level >16.7 mmol/L were allocated into four groups with 3 rats per group:
1. Rats from G2 were subjected to IRI surgery with 60 min I/6 hr R, and D-Cy5 was administrated 1 hr after surgery;
2. Rats from G3 and G4 were subjected to IRI surgery with 45 min I/24 hr R, and D-Cy5 was administrated 1 hr and 12 hr after surgery, respectively;
3. Rats from G1 were subjected to sham operation with similar surgical procedures performed except that artery clamps were not applied. D-Cy5 was administrated 1 hr after surgery.

General Observation & Body Weight Changes

A general observation was performed during the in-life study period. Animals showed clinical symptoms of type 1 diabetes, including increased consumption of drinking water and urine volume. No obvious physical or behavioral abnormalities were observed. Body weight changes and growth curves were monitored. The body weight of animals fluctuated in the first week after STZ application and then increased slowly. The weight growth rate maintained within 10% during the entire study.

Blood Glucose Levels

Blood glucose was measured 4 days post-STZ injection and on the day right before surgery. The blood glucose levels of all rats were above 16.7 mmol/L.

Blood Biochemistry and GFR

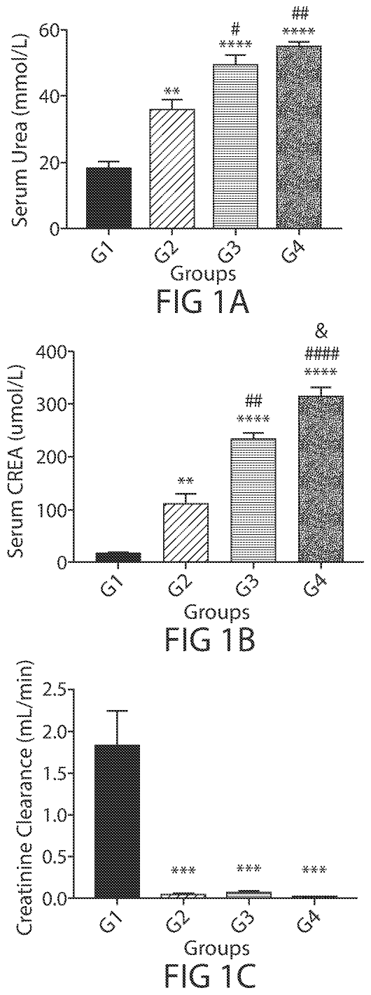

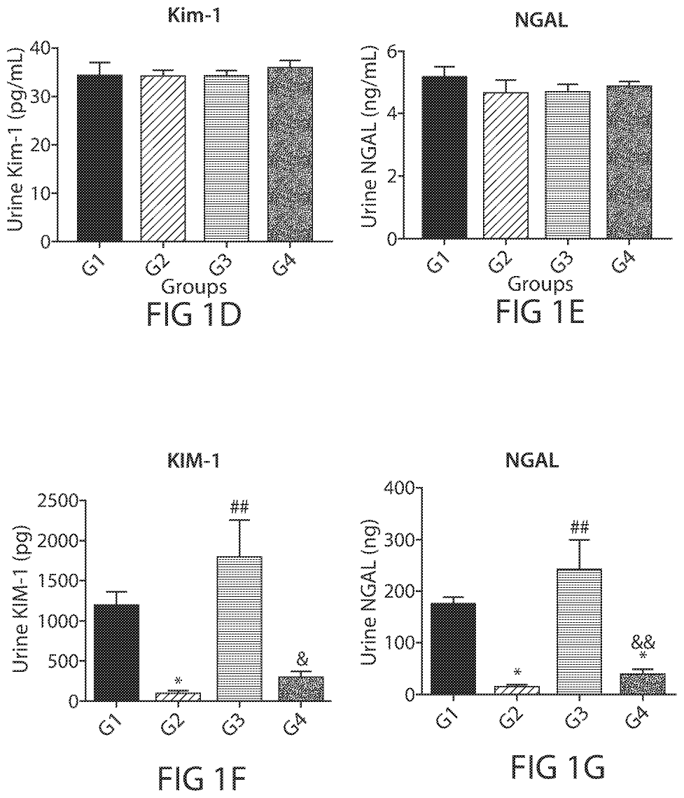

BUN, serum creatinine, urine creatinine levels were measured to assess renal function. As shown in FIGS. 1A-1C, elevated BUN and serum creatinine were observed in model groups (G2-G4). The BUN and serum creatinine levels of G3 and G4 were significantly higher than those of G2. Urine creatinine concentration in G3 and G4 was significantly lower than that of G1. GFR was calculated upon creatinine clearance, and ischemia/reperfusion surgery under both conditions (G2 and G3) resulted in significantly lower GFR compared to the sham-operated group (G1).

NGAL and KIM-1

The urine NGAL and KIM-1 levels were measured using ELISA kits. As shown in FIGS. ID-1G, the NGAL and KIM-1 content in urine samples from G2 and G4 were significantly lower than that of G1 (FIGS. 1F and 1G), while the NGAL and KIM-1 content in urine samples from G3 were higher than that in G1. However, the NGAL and KIM-1 concentration showed no significance between groups (FIGS. 1D and 1E).

Gross Observation of Kidney

After euthanasia, bilateral kidneys were collected and imaged. The kidneys appeared in varying degrees of green. The size of kidneys in G2-G4 was larger than that of G1.

Histopathology Analysis

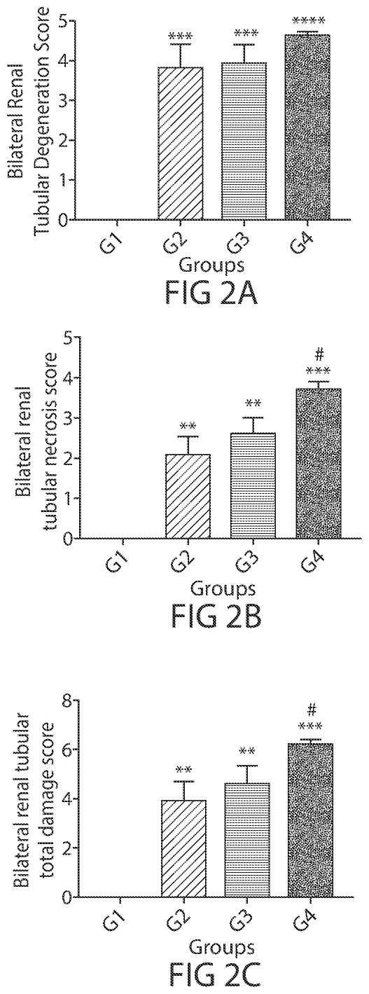

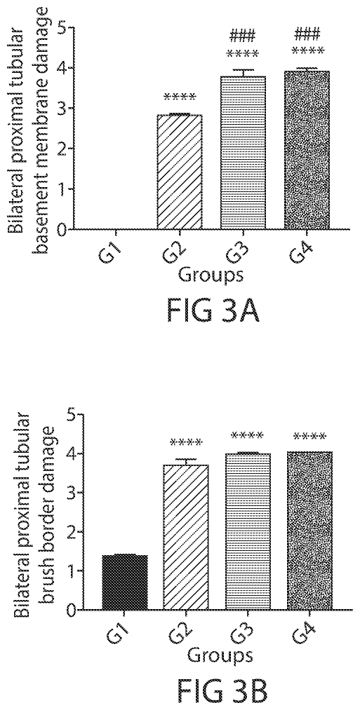

Histopathology analysis was performed using H&E and PAS methods:
H&E. Ischemia/reperfusion-induced varying degrees of renal injury, mainly characterized by epithelial flattening and tubular dilatation (FIGS. 2A-2C). On the contrary, no pathological lesions were observed in the sham group animals.
PAS: Pathological features including dilated renal tubules, detached brush border, and damaged basement membranes were observed from the model groups (FIGS. 3A-3B) but were absent from the sham surgery group.

Colocalization Study

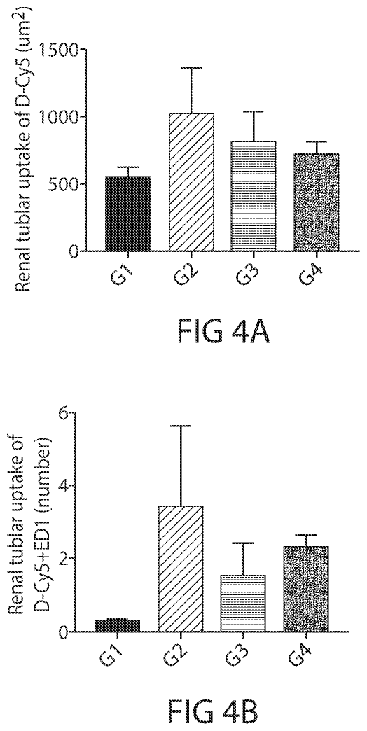

Renal uptake of D-Cy5 and its colocalization with macrophages were evaluated by immunofluorescence (IF) staining. Macrophage was visualized by an anti-CD68 antibody [ED1]. The fluorescence images showed that D-Cy5 positive areas were mainly located in the proximal tubule. The renal uptake of D-Cy5 in G2-G4 groups appeared higher than that in G1 (FIG. 4A). The co-staining results of antibody ED1 and D-Cy5 showed that the number of ED1-positive cells was small. The number of CD68-positive cells in G2-G4 appeared higher than that in G1 (FIG. 4B).

In conclusion, a single i.p. injection of STZ successfully induced type 1 diabetes in Wistar rats. Glucose levels increased to ˜30 mM prior to IRI. Thereafter, bilateral renal IRI and renal dysfunction were induced on this diabetic model. GFR was significantly reduced from 1.8 mL/min (sham) to <0.1 mL/min in IRI rats. Serum creatinine and BUN were significantly elevated in RI groups (G4>G3>G2). The degree of kidney damage increased with the longer reperfusion time prior to sacrifice (G4, G3>G2). The degree of renal injury after 60 min I/6 hr R was slightly less than that after 45 min I/24 hr R. In all IRI groups, renal tubular necrosis was moderate to severe and proximal tubule damage was severe. Maximal uptake of the D-Cy5 was observed in renal tubules in reactive macrophages observed in G2. The uptake of D-Cy5 by renal cells and renal macrophages correlated with the degree of injury.

In AKI and CKD, ischemia in the kidney results in inflammation and tissue damage. Patients with underlying renal dysfunction such as seen in diabetes, are more prone to AKI and CKD. The initial response to injury is the infiltration of reactive macrophages into the kidney with subsequent pro-inflammatory cytokine expression. A novel platform technology, based on hydroxyl dendrimers, enables selective targeting to reactive macrophages in the ischemic kidney upon systemic administration, with renal clearance maximizing kidney exposure. Hydroxyl dendrimers have demonstrated safety at doses up to 40 mg/kg in humans and are recovered intact in the urine. Conjugation of drugs to the hydroxyl dendrimers can enable selective targeting to improve efficacy and safety in treatment of AKI and CKD.

In the current study, a diabetic model of AKI was successfully established to evaluate targeting of hydroxyl dendrimers to reactive macrophages. Prolonged ischemia followed by rapid reperfusion increased reactive macrophages and subsequent uptake of hydroxyl dendrimers. Given the high incidence of diabetic nephropathy and higher risk for acute kidney injury in these patients, these results provided a model and treatment strategy to evaluate targeted therapies with hydroxyl dendrimer drug conjugates to treat AKI and CKD.

Unless defined otherwise, all technical and scientific terms used herein have the same meanings as commonly understood by one of skill in the art. Publications cited herein and the materials for which they are cited are specifically incorporated by reference.

Those skilled in the art will recognize, or be able to ascertain using no more than routine experimentation, many equivalents to the specific embodiments of the aspects described herein. Such equivalents are intended to be encompassed by the following claims.

What is claimed is:

1. A method for treating or preventing one or more symptoms of a kidney injury, disease, and/or disorder in a subject in need thereof, the method comprising:
administering to the subject a formulation comprising dendrimers complexed to, covalently conjugated to, or having intra-molecularly dispersed or encapsulated therein, one or more therapeutic or prophylactic agents,
wherein the dendrimer-agent formulation is administered in an amount effective to treat, alleviate or prevent one or more symptoms of the kidney injury, disease, and/or disorder.

2. The method of claim 1, wherein the kidney injury, disease and/or disorder is an acute or chronic kidney disease.

3. The method of claim 1 or 2, wherein the kidney injury, disease and/or disorder is caused by ischemia/reperfusion injury.

4. The method of claim 3, wherein the kidney injury, disease and/or disorder is caused by a condition selected from the group consisting of infection, sepsis, ischemia-reperfusion injury, diabetic complications, hypertension, obesity, autoimmunity diabetes, high blood pressure, heart failure, kidney disease, liver disease, and cancer.

5. The method of any one of claims 1-4, wherein the dendrimers are hydroxyl-terminated dendrimers.

6. The method of any one of claims 1-5, wherein the dendrimers are generation 4, generation 5, or generation 6 poly(amidoamine) dendrimers.

7. The method of any one of claims 1-6, wherein the therapeutic agent is an anti-inflammatory agent.

8. The method of any one of claims 1-6, wherein the therapeutic agent is a PPAR-δ agonist.

9. The method of claim 8, wherein the PPAR-δ agonist is GW0742, GW0742-amide derivative and GW0742-ester derivative, GW0742-amide derivative, or GW0742-ester derivative.

10. The method of claim 7 where the anti-inflammatory agent is selected from the group consisting of N-acetylcysteine, steroidal anti-inflammatory drugs, non-steroidal drugs, cyclosporine, tacrolimus, rapamycin, SGLT2 inhibitors, LPA1 receptor antagonists, vasopression V2-receptor antagonists, endothelian receptor antagonists, and uric acid transporter inhibitors.

11. The method of any one of claims 1-10, wherein the formulation is administered in an amount effective to reduce inflammation in the kidney.

12. The method of any one of claims 1-10, wherein the formulation is administered in an amount effective to reduce tubular damage, tubular epithelial flattening, tubular dilatation, and tubular epithelial cell necrosis, and/or apoptosis in the kidneys.

13. The method of any one of claims 1-10, wherein the formulation is administered in an amount effective to reduce serum levels of creatinine and/or blood urea nitrogen (BUN); to reduce urine NGAL and/or KIM-1 content; and/or to improve glomerular filtration rate (GFR).

14. The method of any one of claims 1-10, wherein the formulation is administered in an amount effective to reduce the amount or presence of one or more pro-inflammatory cells, chemokines, and/or cytokines in the kidney.

15. The method of claim 14, wherein the formulation is administered in an amount effective to reduce one or more pro-inflammatory cytokines selected from the group consisting of TNF-α, TFN-γ, IL-6, IL-1β, IL-23, and IL-17.

16. The method of any one of claims 1-15, wherein the formulation comprises a therapeutic, prophylactic or diagnostic agent selected from the group consisting of chemotherapeutic agents, anti-angiogenic agents, anti-excitotoxic agents, inhibitors of glutamate formation/release, anti-VEGF agents including aflibercept, immunomodulators such as rapamycin, uric acid transporter (URAT1) inhibitors, vasopression V2-receptor antagonists, endothelin receptor antagonists, subtype 2 sodium-glucose transport protein (SGLT2) inhibitor, and LPA1 receptor antagonists.

17. The method of any one of claims 1-16, wherein the formulation is formulated for intravenous, subcutaneous, or intramuscular administration.

18. The method of any one of claims 1-16 wherein the formulation is formulated for enteral administration.

19. The method of any one of claims 1-16, wherein the formulation is administered via the intravenous, subcutaneous, or intramuscular route.

20. The method of any one of claims 1-19, wherein the formulation is administered prior to, in conjunction, subsequent to, or in alternation with treatment with one or more additional therapies or procedures.

21. The method of claim 20, wherein the one or more additional procedures include administering intravenous fluids and/or hemodialysis.

22. A pharmaceutical formulation for use in the method of any one of claims 1-21.

23. A kit comprising
(1) one or more single unit dose of a composition comprising dendrimers covalently conjugated with one or more PPAR-δ agonists, and
(2) instructions on how the dose is to be administered for treatment of one or more kidney injuries, diseases, and/or conditions.

24. A composition comprising a compound that comprises a dendrimer conjugated to a PPAR-δ agonist through an ester, ether, or amide linkage, wherein the dendrimer comprises a high density of surface hydroxyl groups.

25. The composition of claim 24, wherein the PPAR-δ agonist is conjugated to the ester, ether, or amide linkage through a spacer.

26. The composition of claim 25, wherein the spacer comprises alkyl groups, heteroalkyl groups, or alkylaryl groups.

27. The composition of claim 25 or 26, wherein the spacer comprises a peptide.

28. The composition of any one of claims 25-27, wherein the spacer comprises polyethylene glycol.

29. The composition of any one of claims 24-28, wherein conjugation of the PPAR-δ agonist occurs on less than 50% of total available surface functional groups of the dendrimer prior to the conjugation.

30. The composition of any one of claims 24-29, wherein conjugation of the PPAR-δ agonist occurs on less than occurs on less than 5%, less than 10%, less than 20%, less than 30%, or less than 40% of total available surface functional groups of the dendrimer prior to the conjugation.

31. The composition of any one of claims 24-30, wherein the PPAR-δ agonist is an indanylacetic acid derivative.

32. The composition of any one of claims 24-31, wherein the PPAR-δ agonist is GW0742.

33. The composition of any one of claims 24-32, wherein the PPAR-δ agonist is a GW0742-amide derivative or a GW0742-ester derivative.

34. The composition of any one of claims 24-33, wherein the dendrimer comprises poly(amidoamine), polypropylamine (POPAM), polyethylenimine, polylysine, polyester, iptycene, aliphatic poly(ether), and/or aromatic polyether.

35. The composition of any one of claims 24-34, wherein the dendrimer is a poly(amidoamine) dendrimer.

36. The composition of any one of claims 24-35, wherein the dendrimer is a generation 4, generation 5, or generation 6 poly(amidoamine) dendrimer.

37. The composition of any one of claims 24-36, wherein the zeta potential of the compound is between −25 mV and 25 mV.

38. The composition of any one of claims 24-37, wherein the zeta potential of the compound is between −20 mV and 20 mV, between −10 mV and 10 mV, between −10 mV and 5 mV, between −5 mV and 5 mV, or between −2 mV and 2 mV.

39. The composition of any one of claims 24-38, wherein the surface charge of the compound is neutral or near-neutral.

40. The composition of any one of claims 24-39, wherein the dendrimer conjugated to the PPAR-δ agonist through an ether or amide linkage.

41. The composition of any one of claims 24-40, wherein the dendrimer conjugated to the PPAR-δ agonist through an ether linkage.
